(19) United States
(12) Patent Application Publication
(10) Pub. No.: US 2025/0339392 A1
(43) Pub. Date: Nov. 6, 2025

(54) THE USE OF LRRC8 PROTEIN MODULATORS TO PREVENT AND TREAT CARDIOVASCULAR DISEASE

(71) Applicants: Washington University, St. Louis, MO (US); Senseion Therapeutics, Inc., St. Louis, MO (US)

(72) Inventors: Rajan Sah, St. Louis, MO (US); Daniel J. Lerner, St. Louis, MO (US); Jorge Di Paola, St. Louis, MO (US)

(21) Appl. No.: 18/854,209

(22) PCT Filed: Apr. 6, 2023
(86) PCT No.: PCT/US2023/017744

§ 371 Date: Oct. 4, 2024

Related U.S. Application Data
(60) Provisional application No. 63/328,010, filed on Apr. 6, 2022.

(51) Int. Cl. A61K 31/192; A61K 31/138; A61K 31/41; A61P 7/02; A61P 7/04

(52) U.S. Cl. CPC A61K 31/192; A61K 31/138; A61K 31/41; A61P 7/02; A61P 7/04

(57) ABSTRACT

The present invention is directed to various methods of for preventing and/or treating cardiovascular thrombosis, hereditary or acquired defects in platelet function, and hereditary or acquired thrombocytosis, wherein the method comprises administering a therapeutically effective amount of an LRRC8 protein modulator to the patient. Compounds that are LRRC8 protein modulators are also described, including DCPIB (4-[2[butyl-6,7-dichloro-2-cyclopentyl-2,3-dihydro-1-oxo-1H-inden-5-yl)oxy]butanoic acid).

### FIELD OF THE INVENTION

The present invention is directed to various methods of suppressing cardiovascular thrombosis and platelet dysfunction. For example, the present invention relates to various methods of preventing or treating a disease in which cardiovascular thrombosis contributes to the disease state such as myocardial ischemia and infarction, stroke, transient cerebrovascular ischemia, peripheral arterial disease, thromboembolism associated with atrial fibrillation and flutter, and thromboembolism associated with the use of cardiovascular medical devices, as well as, disease related to platelet dysfunction including abnormal adhesion, aggregation, activation, or thrombosis.

### BACKGROUND OF THE INVENTION

The family of LRRC8 (leucine-rich repeat-containing protein 8) protein channels are comprised of different heterohexameric combinations of one or more of five LRRC8 monomer proteins (LRRC8 A-E). The genes encoding the five LRRRC8 monomer proteins are located on three human chromosomes: 1 (LRRC8 B, LRRC8 C, and LRRC8 D), 9 (LRRC8 A), and 19 (LRRC8 E).

Platelets are megakaryocyte cell fragments that are integral to in vivo thrombosis required for hemostasis. A variety of agonist molecules including thrombin, thromboxane, adenosine diphosphate (ADP), and collagen stimulate platelet activation including shape change and fusion of lysosomal-related organelles (LRO) alpha granules, dense granules, and lysosomes) with the platelet plasma membrane. This membrane fusion results in expression of adhesion molecules on the platelet surface, and release of pro-coagulant molecules including von Willebrand factor and fibrinogen (alpha granules) and ATP, ADP, Ca²⁺, and serotonin (dense granules) that permit the platelet to adhere to the de-endothelialized surface of blood vessels and activate coagulation pathways, leading to platelet aggregation and thrombus formation.

Thrombus formation is integral to hemostasis after blood vessels have been compromised by trauma or surgical manipulation. Thrombosis can also cause significant morbidity and mortality when it occurs on the surface of ruptured atherosclerotic plaques, the left atrial appendage, or medical devices with surfaces exposed to flowing blood. In situ thrombus formation on ruptured arterial atherosclerotic plaque can cause myocardial, cerebrovascular, and lower limb ischemia and infarction. Embolization of thrombus formed in the left atrial appendage of patients with atrial fibrillation and flutter can cause cerebrovascular ischemia and infarction as well as myocardial and lower limb ischemia and infarction. Thrombus formation on the surface of medical devices exposed to flowing blood in vivo (e.g. prosthetic cardiac valves, coronary artery diagnostic and therapeutic catheters; coronary, cerebrovascular, and peripheral stents; intravascular blood flow pumps) or ex vivo (e.g. extracorporeal membrane oxygenation (ECMO) devices; external ventricular assist devices) can lead to blood conduit occlusion and embolization causing myocardial, cerebrovascular and lower limb ischemia and infarction.

A number of pharmacologic strategies have been developed to prevent thrombosis that causes morbidity and mortality, including inhibitors of platelet enzymes that participate in enzymatic catalysis of thromboxane formation (e.g. acetyl salicylic acid inhibits cyclo-oxygenase), inhibitors of platelet agonists, (e.g. bivalirudin inhibits the proteolytic site on thrombin), and inhibitors of platelet receptors for platelet agonists (e.g. clopidogrel inhibits P2Y12 purinergic receptor). Despite these therapies, very significant residual risk for morbidity and mortality from cardiovascular thrombosis persists. In the US it is estimated there are 605,000 new and 200,000 recurrent myocardial infarctions per year and 610,000 new and 185,000 recurrent strokes per year. In addition, significant morbidity and mortality from thrombosis associated with the use of cardiovascular medical devices persists including the use of intravascular diagnostic and treatment intravascular catheters, intravascular blood pumps, ventricular assist devices, intra-aortic balloon pumps, extracorporeal membrane oxygenation devices, prosthetic cardiac valves, and cardiac implantable electronic devices.

The LRRC8 protein family has five members encoded by genes on 3 chromosomes: LRRC8A on human chromosome 9; LRRC8B, LRRC8C and LRRC8D on human chromosome 1; and LRRC8E on human chromosome 19. These proteins can form hetero-hexamers that can function as membrane channels for ions and small molecules. LRRC8A was discovered in a child with a gammaglobulinemia associated with a chromosomal translocation that truncated this protein. In 2014 it was shown that the Voltage Regulated Anion Current (VRAC) was mediated by a hexameric LRRC8 protein that included at least LRRC8A. Subsequently, it was shown that LRRC8 proteins are expressed in tissue-specific combinations. In 2020, it was demonstrated that LRRC8A was required for normal lysosomal function in multiple cell lines including HAP1 cells derived from KBM-7 cells taken from a patient with chronic myelogenous leukemia. An essential step in platelet activation is fusion of the lysosomal-related organelles, including alpha-granules, dense-granules, and lysosomes with the plasma membrane.

Increased platelet volume has been associated with myocardial infarction and death following myocardial infarction, worse outcome following acute ischemic stroke, and peripheral arterial disease. Increased platelet volume has also been associated with worse clinical outcomes following primary percutaneous coronary revascularization, type 2 diabetes, microvascular complications of diabetes, and nonalcoholic fatty liver disease.

Previously, it has been shown that LRRC8 proteins regulate cell volume in adipocytes and other cells. Recently, it has been shown that small molecules including but not limited to DCPIB (4-((2-Butyl-6,7-dichloro-2-cyclopentyl-1-oxo-2,3-dihydro-1H-inden-5-yl)oxy)butanoic acid) can regulate LRRC8 function in vivo to regulate glycemic control and other functions.

Cardiovascular disease (CVD) and Type 2 diabetes (T2D) are overlapping global pandemics. It is estimated there are 463 million adults with T2D globally, and that 30-34% of these, or 149 million people, have both T2D and CVD. T2D accelerates the development and severity of CVD, increases the risk of coronary death, non-fatal myocardial infarction (MI), and ischemic stroke 2-4 fold, and worsens clinical outcomes of patients that have CVD events. CVD is the most common cause of death in patients with T2D—about two-thirds of patients with DM die of CVD. Stroke and MI most often occur when a platelet-rich thrombus forms at the site of a ruptured atherosclerotic plaque, partially or totally occluding the vessel lumen, resulting in downstream ischemia. In T2D, multiple factors accelerate this process including a prothrombotic state with hyper-reactive platelets and increased circulating coagulation factors, as well as, abnormal vessel wall function including decreased endothelial nitric oxide synthase (eNOS) signaling. In addition, multiple factors make platelets in T2D patients less responsive to inhibition by current antiplatelet agents. The economic burden of caring for stroke and myocardial ischemia in patients with T2D is staggering. In 2018, patients with T2D in the US had 1.87 million hospitalizations for major CVD including 440,000 for ischemic heart disease and 334,000 for stroke, and 154,000 for a lower extremity amputation. It is estimated 49% of the direct cost of care for T2D is for the prevention and treatment of CVD. The total direct cost of care for T2D in the US increased from $188 billion in 2012 to $237 billion in 2017, an increase of 26%, and a meta-analysis of studies from multiple countries indicates the direct cost of CVD care in patients with T2D may account for up to 49% of the total direct care costs. There are at least ten classes of drugs approved to treat hyperglycemia associated with T2D. While newer glycemic control agents like SGLT2 inhibitors and GLP1 agonists can help reduce CVD events in T2D, significant residual CVD risk remains. Currently there are four classes of drugs commonly used to prevent and/or treat MI or stroke: aspirin, P₂Y₁₂ receptor inhibitors, thrombin receptor inhibitors, and platelet aIIbb3 inhibitors. They have been used alone as monotherapy and in combination (dual-antiplatelet therapy; DAPT) to prevent and treat stroke and MI. While current antiplatelet drugs can reduce CVD events and death, their therapeutic potential is limited by major bleeding (e.g. intracranial hemorrhage and bleeding death) associated with use.

There is an urgent need for therapies to prevent and treat diseases related to cardiovascular thrombosis including myocardial ischemia and infarction, stroke, transient cerebrovascular ischemia, peripheral arterial disease, thromboembolism associated with atrial fibrillation and flutter, and thromboembolism associated with the use of cardiovascular medical devices including intravascular diagnostic and treatment intravascular catheters, intravascular blood pumps, ventricular assist devices, intra-aortic balloon pumps, extracorporeal membrane oxygenation devices, prosthetic cardiac valves, and cardiac implantable electronic devices. Additionally, there is a large unmet clinical need for a drug that improves glycemic control in T2D and also safely prevents cerebral and coronary vascular thrombosis. This unmet clinical need represents a large commercial opportunity. It is estimated there are more than 8 million patients with T2D that are eligible for secondary prevention of an MI or stroke with antiplatelet drugs according to current guidelines. Using current prices for new oral antiplatelet drugs (e.g. ticagrelor) as a benchmark, this corresponds to a >$20 billion annual market just for secondary MI prevention in only the US and EU.

### BRIEF SUMMARY

Various aspects of the present invention are directed to methods of preventing or treating thrombosis. In various embodiments, methods for preventing or treating thrombosis in a subject in need thereof comprises administering to a subject a therapeutically effective amount of DCPIB (4-((2-Butyl-6,7-dichloro-2-cyclopentyl-1-oxo-2,3-dihydro-1H-inden-5-yl)oxy)butanoic acid) or a congener thereof. In some embodiments, the method comprises administering to a subject a therapeutically effective amount of a compound selected from the group consisting of:

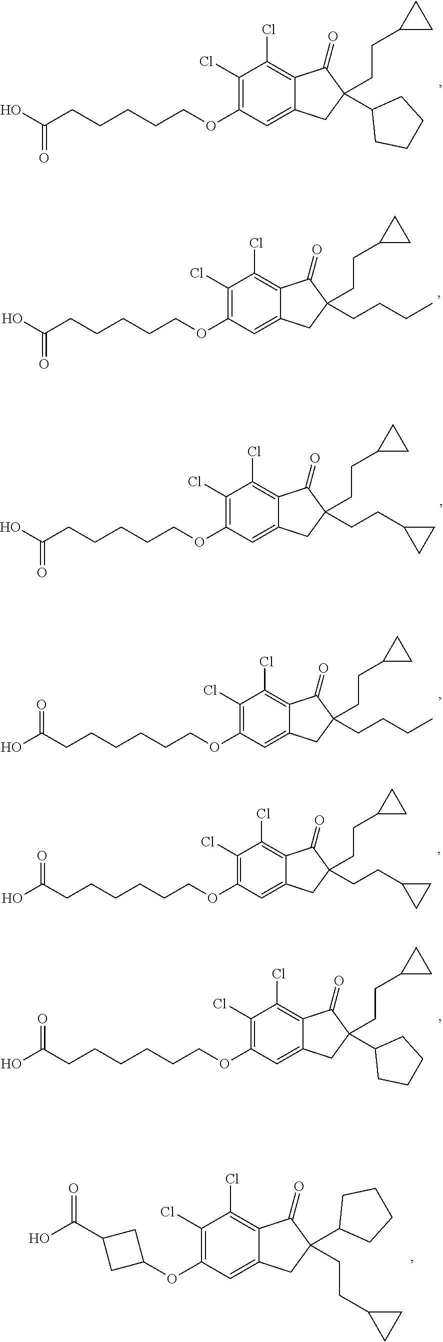

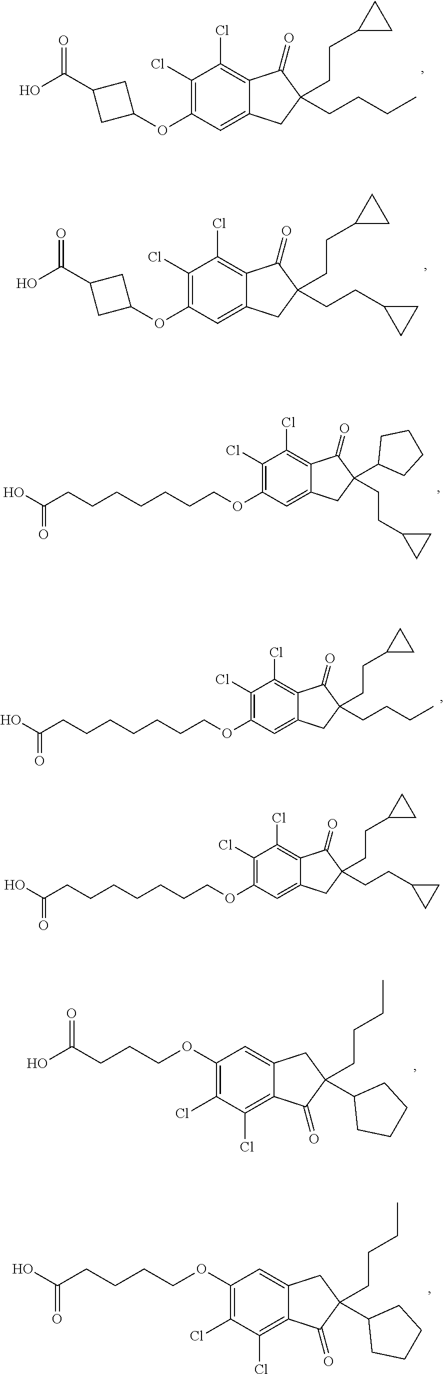

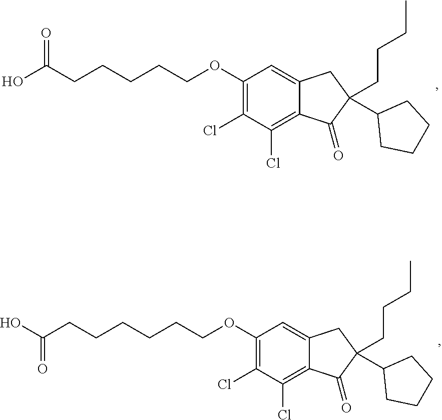 [SMILES: CCCCC1(C2CCCC2)Cc2cc(OCCCCCC(=O)O)c(Cl)c(Cl)c2C1=O ; CCCCC1(C2CCCC2)Cc2cc(OCCCCCCC(=O)O)c(Cl)c(Cl)c2C1=O]

and salts and geometric isomers thereof.

In various embodiments, the methods comprise administering to a subject a therapeutically effective amount of a compound of Formula (I), and salts and geometric isomers thereof:

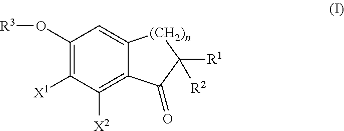

wherein
R¹ and R² are each independently hydrogen, substituted or unsubstituted alkyl, substituted or unsubstituted alkenyl, substituted or unsubstituted alkoxy, substituted or unsubstituted aryl, substituted or unsubstituted heteroaryl;
R³ is —Y—C(O)R⁴, Z—N(R⁵)(R⁶), or —Z-A;
A is selected from the group consisting of:

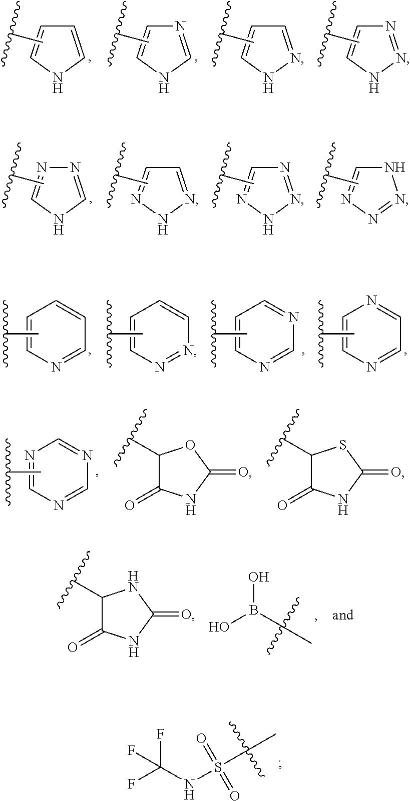

R⁴ is hydrogen, substituted or unsubstituted alkyl, —OR⁷, or —N(R⁸)(R⁹);
X¹ and X² are each independently substituted or unsubstituted alkyl, halo, —OR¹⁰, or —N(R¹¹)(R¹²);
R⁵, R⁶, R⁷, R⁸, R⁹, R¹⁰, R¹¹, and R¹² are each independently hydrogen or substituted or unsubstituted alkyl;
Y and Z are each independently a substituted or unsubstituted carbon-containing moiety having at least 2 carbon atoms; and n is 1 or 2.

In various embodiments, the methods of preventing or treating thrombosis include treating a disease in which thrombosis contributes to the disease state, the present invention relates to various methods of preventing or treating a disease in which cardiovascular thrombosis contributes to the disease state such as myocardial ischemia and infarction, stroke, transient cerebrovascular ischemia, peripheral arterial disease, thromboembolism associated with atrial fibrillation and flutter, and thromboembolism associated with the use of cardiovascular medical devices, as well as, disease related to platelet dysfunction including abnormal adhesion, aggregation, activation, or thrombosis.

Other objects and features will be in part apparent and in part pointed out hereinafter.

### BRIEF DESCRIPTION OF THE FIGURES

FIG. 1 depicts how LRRC8 proteins regulate human platelet volume. Single-nucleotide polymorphisms (SNPs) assigned to LRRC8 genes A-D are associated with human platelet volume in databases of human genome-wide association studies (GWAS), human phenome-wide association studies (PheWAS), and expression quantitative loci (eQTL) in whole blood. Genome-wide association studies (GWAS) indicate LRRC8 proteins regulate human platelet volume. A search of the NHGARI-EBI Catalog of eligible published human genome-wide association studies (accessed 9 Feb. 2022) was conducted and associations were identified between platelet volume and 6 SNP variants assigned to three genes on two chromosomes (LRRC8 A (2×10⁻¹⁰) on 9q34.11 and LRRC8 C (5×10⁻²³ & 3×10⁻¹⁴) and LRRC8 D (4×10⁻¹³ & 6×10⁻¹²) on lp22.2). Phenome-wide association studies (PheWAS) indicate LRRC8 proteins regulate human platelet volume. After identifying 6 SNPs associated with human platelet volume in the NHGARI-EBI Catalog of published human GWAS studies, the Common Metabolic Diseases Knowledge Portal (accessed 3 Mar. 2022) and the PheWAS-IEU OpenGWAS Project catalog (PheWAS Catalog) were searched for PheWAS associations between these 6 SNP variants and platelet volume (8.9×10⁻¹⁰, 2.3×10⁻¹⁰, 5.7×10⁻¹⁸, 7.9×10⁻¹⁷, 1.5×10⁻⁶ and 1.4×10⁻⁹), as well as, 4 additional SNP variants assigned to LRRC8 B, the interval of overlap between LRRC8 B and C, and LRRC8 C that were initially identified because they are associated with Blood Monocyte Count or Diastolic Blood Pressure in the GWAS catalog. In all, the Platelet Volume phenotype is associated with 10 SNPs assigned to 4 LRRC8 genes (LRRC8 A-D) on 2 chromosomes (1p22.2 & 9q34.11). eQTL studies indicate LRRC8 proteins regulate human platelet volume. The GTEX Portal database expression quantitative trait loci (eQTL) database (accessed 5 Mar. 2022) was then searched and significant associations were found between levels of mRNA transcripts for LRRC8B (P=2.8×10⁻⁵⁶) and LRRC 8C (P=4.8×10⁻⁶³) in whole blood and SNPs assigned to those genes that were associated with platelet volume in the NHGARI-EBI Catalog of published human GWAS studies.

FIG. 2 depicts how LRRC8 proteins regulate human platelet count. Single-nucleotide polymorphisms (SNPs) assigned to LRRC8 genes A-D are associated with human platelet count in databases of human genome-wide association studies (GWAS), human phenome-wide association studies (PheWAS), and expression quantitative loci (eQTL) in whole blood. Phenome-wide association studies (PheWAS) indicate LRRC8 proteins regulate human platelet count. The Common Metabolic Diseases Knowledge Portal (accessed 3 Mar. 2022) was searched for PheWAS associations between platelet count and the 13 SNP variants with P-values <10⁻⁵ (accessed 2 Sep. 2022). PheWAS identified an association between 3 SNP variants and platelet count (4.9×10⁻¹¹, 2.6×10⁻¹⁰, and 3.8×10⁻⁷). In all, the platelet count phenotype is associated with 3 SNPs assigned to 2 LRRC8 genes (LRRC8 C-D) on 1 chromosome (lp22.2). eQTL studies indicate LRRC8 proteins regulate human platelet count. The GTEX Portal database expression quantitative trait loci (eQTL) database (accessed 5 Mar. 2022) was searched and significant associations were found between levels of mRNA transcripts for LRRC 8C (P=4.8×10⁻⁶³) in whole blood and a SNP assigned to LRRC 8C that was associated with platelet count in the Common Metabolic Diseases Knowledge Portal for (accessed 3 Mar. 2022) or the PheWAS-IEU OpenGWAS Project catalog (PheWAS Catalog).

FIG. 3 depicts how LRRC8A-D mRNA transcripts are expressed in human platelets at high levels compared to other mRNA transcripts. The Plateletomics Interactive Results Tool version 1.0 to search the Plateletomics database (accessed 5 Mar. 2022) was used, and it was found that mRNA transcripts for LRRC 8A-8D are expressed in human platelets and that they are in the top 76^{th}, 14^{th}, 43^{rd}, and top 5^{th} percentile, respectively, of all mRNA transcripts detected in the platelets of 154 patients. This indicates mRNA transcripts for LRRC 8A-D are highly expressed in platelets compared to other mRNA transcripts.

FIG. 4 depicts how human platelets contain mRNA transcripts for LRRC 8A, LRRC 8B, LRRC 8C, and LRRC8D in proportions of 1 to 2.5 to 1.5 to 3.0, respectively. LRRC 8A-E transcripts were searched for in the database in Supplementary Table S2A from Simon, L. M. et al. (Blood 123, e37-45 (2014)), and it was found that LRRCA, LRRC 8B, LRRC 8C, and LRRC 8D mRNA transcripts are expressed in human platelets, and that LRRC 8B, LRRC8C, and LRRC8D are expressed at approximately 2.5 fold, 1.5 fold, and 3-fold the levels of LRRC 8A mRNA, respectively.

FIG. 5 depicts how the degree of human platelet aggregation stimulated by protease-activated receptor 4 (PAR4) agonist is proportional to LRRC8A expression. The Plateletomics Interactive Results Tool version 1.0 was used to search the Plateletomics database (accessed 5 Mar. 2022), and data was found that demonstrated that the intensity of human platelet aggregation stimulated by protease-activated receptor 4-activating peptide (PAR4 AP), an agonist for the platelet PAR4 thrombin receptor, is significantly associated with the amount of LRRC8A mRNA expressed in human platelets.

FIG. 6 depicts how the degree of human platelet aggregation stimulated by adenosine diphosphate (ADP) and protease-activated receptor 1 (PAR1) agonist are inversely proportional to LRRC8B expression. The Plateletomics Interactive Results Tool version 1.0 was used to search the Plateletomics database (accessed 5 Mar. 2022), and data was found that demonstrated that the intensity of human platelet aggregation stimulated by adenosine diphosphate (ADP), an agonist for the platelet P2Y12 and other ADP receptors, and by protease-activated receptor 1-activatin peptide (PAR1 AP), an agonist for the platelet PAR1 thrombin receptors, are significantly inversely associated with the amount of LRRC8B mRNA expressed in human platelets.

FIG. 7 depicts how the degree of human platelet aggregation stimulated by protease-activated receptor 4 (PAR4) agonist is inversely proportional to LRRC8C expression. The Plateletomics Interactive Results Tool version 1.0 was used to search the Plateletomics database (accessed 5 Mar. 2022), and data was found that demonstrated that the intensity of human platelet aggregation stimulated by protease-activated receptor 4-activating peptide (PAR4 AP), an agonist for the platelet PAR4 thrombin receptor, is associated inversely associated with the amount of LRRC8C mRNA expressed in human platelets.

FIG. 8 depicts how the degree of human platelet aggregation stimulated by protease-activated receptor 1 (PAR1) agonist is inversely proportional to LRRC8D expression. The Plateletomics Interactive Results Tool version 1.0 was used to search the Plateletomics database (accessed 5 Mar. 2022), and data was found that demonstrated that the intensity of human platelet aggregation stimulated by protease-activated receptor 1-activating peptide (PAR1 AP), an agonist for the platelet PAR1 thrombin receptor, is inversely associated with the amount of LRRC8D mRNA expressed in human platelets.

FIG. 9 depicts how there are racial differences in LRRC8A-D expression in human platelets. The Plateletomics Interactive Results Tool version 1.0 to search the Plateletomics database (accessed 5 Mar. 2022), and data was found that demonstrated that LRRC8C and LRRC8D mRNA expression is significantly lower in people identified as Black compared to people identified as White. LRRC8A and LRRC8B gene expression are also lower in people identified as Black compared to people identified as White, but did not exceed the threshold for significance in this dataset.

FIG. 10 depicts how multiple regression analyses demonstrate LRRC8A, LRRC8B, and LRRC8D play significant roles in human platelet aggregation in response to ADP, PAR1 agonist peptide, and PAR4 agonist peptide. Regression coefficients for LRRC 8A-D in multiple regression analyses for platelet aggregation response in 5911 commonly expressed mRNAs in human platelets from 154 human subjects from the 2014 Supplementary Table S3 of Simon, L. M. et al. (Blood 123, e37-45 (2014)), and the corresponding values for tests of significance demonstrate that the degree of ADP-induced platelet aggregation is significantly associated with the level of LRRC8A mRNA and the inverse of the level of LRRC8B mRNA; the degree of PAR1-induced platelet aggregation is significantly associated with the inverse of the level of LRRC8B and LRRC8D mRNA; and the degree of PAR4-induced platelet aggregation is associated with the level of LRRC8D mRNA. The regression specification dependent variable was the degree of platelet aggregation in response to one of four platelet agonists (ADP, PAR1 AP, PAR 2AP, and arachidonic acid), and the independent variables were age, race, gender, platelet number, and BMI, and the levels of 5,911 commonly expressed mRNAs.

FIGS. 11A and 11B depict how LRRC8A in platelets regulates platelet volume in vivo. Whole blood was collected from the inferior vena cava of 20-week old WT mice and mice lacking platelet LRRC8A into heparin anticoagulant (15 USP units/mL), and a whole blood CBC was run on an Element HT5 veterinary hematology analyzer (Heska, Loveland) which demonstrated the mean platelet volume (MPV) of platelets lacking LRRC8A was significantly greater than the MPV of WT platelets (P<0.01; unpaired, two-sided t-test). The platelet count was also increased in the mice with platelets lacking LRRC8A compared to WT mice, but the difference did not cross the threshold for significance (P=00.06).

FIG. 12 depicts how LRRC8A is required for normal platelet adhesion of human platelets to collagen. Whole blood was collected from the inferior vena cava of 20-week old WT mice and mice lacking platelet LRRC8A into heparin anticoagulant. The heparinized whole blood was fluorescently labeled with DiOC6 (3,39-dihexyloxacarbocyanine iodide). The whole blood was perfused through a polydimethysiloxane (PDMS) microfluidic device plasma bonded to a glass slide patterned with bovine type 1 collagen at a constant upstream pressure for 5 minutes followed by PBS for 5 minutes. Platelet accumulation was captured using an Olympus IX83 inverted microscope and the surface area coverage of platelets quantified using ImageJ. It was found that absence of LRRC8A significantly reduced platelet adhesion (P<0.0001; two-tailed paired t-test).

FIG. 13 depicts how inhibition of LRRC8 with SN-401 (DCPIB) significantly inhibited adhesion of human platelets to collagen. Human blood from 4 donors was collected into heparin anticoagulant, pooled and incubated with the fluorescently label DiOC6 (3,39-dihexyloxacarbocyanine iodide; Invitrogen) and either 10 μM SN-401 (DCPIB) or vehicle (DMSO) for 10 minutes. The whole blood was perfused through a polydimethysiloxane (PDMS) microfluidic device plasma bonded to a glass slide patterned with bovine type 1 collagen at a constant upstream pressure for 5 minutes followed by PBS for 5 minutes. Platelet accumulation was captured using an Olympus IX83 inverted microscope and the surface area coverage of platelets quantified using ImageJ. It was found that SN-401 significantly reduced adhesion of human platelets to collagen (vehicle v. SN-401; P<0.0001; ordinary one-way ANOVA with Turkeys multiple comparison tests).

FIG. 14 depicts how inhibition of LRRC8 with SN-401 (DCPIB) significantly inhibited thrombosis in vivo. To determine if acute inhibition of LRRC8 channel activity affected platelet function in vivo, vehicle or the known LRRC8 channel inhibitor SN-401 (DCPIB) was administered to 25 to 30-week old C57BL/6 mice by IP injection, and in vivo thrombosis was quantified using an assay for tail wound thrombosis. Specifically, 20 minutes following IP injection with vehicle or 10 mg/kg SN-401, mouse tails were amputated 5 mm from the tail tip, placed in normal saline at 37° C. for 15 minutes, and the relative blood loss during that period was quantified by estimating the hemoglobin concentration in the normal saline by measuring light absorbance at 550 nm. It was found that the mean amount of tail blood loss in mice receiving SN-401 was 4-fold the blood loss in mice receiving vehicle (P<0.05; unpaired two-tailed t-test). This demonstrated pharmacologic inhibition of LRRC8 inhibited thrombosis in vivo.

FIG. 15 depicts how LRRC8 mRNA are highly expressed in human platelets. Percentile expression of platelet LRRC8A-D relative to all platelet transcripts is shown. Data is from the Plateletomics database.

FIG. 16 depicts a Western blot of LRRC8A, GAPDH (loading control) for LRRC8A, p3 integrin, and associated GAPDH for in WT and LRRC8A cKO platelets.

FIG. 17A-17E depicts how megakaryocyte (MK) patch-clamp reveals a robust swell-activated current. FIG. 17A depicts a murine MK perforated patch-clamp. FIG. 17B depicts current density over time in response to swelling with hypotonic solution (HYPO, 205 mOsm), and inhibition by 10 μM SN401. FIG. 17C depicts the current-voltage relationship in WT MK. FIG. 17D depicts the current-voltage relationship in cKO MK. FIG. 17E depicts mean outward and inward currents at +100 mV and −100 mV respectively, in WT, WT+SN401, and cKO MK. * p<0.05;** p<0.01.

FIG. 18 depicts mean platelet volume for WT, LRRC8A cKO, and PF4-Cre;LRRC8Afl/+ controls. **** p<0.001.

FIG. 19A-19B depict how platelets lacking LRRC8A exhibit impaired aggregation and adhesion. FIG. 19A depicts impaired platelet adhesion to collagen coated microfluidic chambers. 2-way Anova, P=0.01. FIG. 19B depicts platelet aggregation in WT and cKO mice in response thrombin (n=7, each group). **** p<0.001.

FIG. 20A-20B depict how platelet-specific LRRC8A ablation impairs platelet activation. FIG. 20A depicts P-selectin exposure in response to thrombin in WT and cKO mice. **** p<0.001. FIG. 20B depicts αIIbβ3 activation in response to thrombin in WT and cKO mice. * p<0.05; **** p<0.001.

FIG. 21A-21B depict how LRRC8A cKO mice exhibit impaired thrombosis in vivo, in FeCl₃-induced carotid injury models. FIG. 21A depicts carotid artery tracings of WT and LRRC8A cKO mice after injury. FIG. 21B depicts time to total occlusion (TITO). *** p<0.001.

FIG. 22A-22C depict how LRRC8A is required for platelet thrombus formation but not fibrin generation in laser-induced cremaster arteriolar thrombosis. FIGS. 22A-22C depict intravital microscopy with WT control and LRRC8A cKO mice. After laser-induced cremaster arteriolar injury, platelet accumulation and fibrin generation were detected by injection of DyLight 649-conjugated anti-CD42c and Alexa 488-conjugated anti-fibrin antibodies, respectively. FIG. 22A depicts representative images. FIG. 22B depicts quantification of the median integrated fluorescence intensities of anti-CD42c (F platelet) antibody signals following laser injury. FIG. 22C depicts quantification of the median integrated fluorescence intensities of anti-fibrin (F fibrin) antibody signals following laser injury.

FIG. 23A-23B depict how SN-401 impairs human platelet adhesion under physiologic shear-flow conditions in collagen-coated microfluidic flow chambers. n=4 blood draws from 4 subjects on 2 separate days. FIG. 23A depicts the percentage of surface area covered by adherent platelets over time in human whole-blood treated with vehicle or 10 μM SN-401. p=0.1 for 2-way Anova. FIG. 23B depicts the area under the curve (AUC) calculated from FIG. 23A for vehicle and SN-401. p=0.1 for t-test.

FIG. 24 depicts how SN-401 treatment increases mouse tail bleeding. Mice were pretreated with Vehicle or SN401 (10 mg/kg i.p×1) and tail bleeding quantified after 15 minutes by measuring absorbance of hemoglobin at 550 nm. * p<0.05.

FIG. 25A-25D depict how congeners SN-418 and SN-418C are potent inhibitors of swell-activated currents recorded from HEK cells. FIG. 25A depicts the chemical structure of SN-418. FIG. 25B depicts the chemical structure of SN-418C. FIG. 25C depicts currents in the presence of varying concentrations of SN-418 and SN-401 normalized to the currents activated in hypotonic solution alone. Lines show the best fits to a logistic equation with IC50 of 420 nM and 4.0 μM for SN-418 and SN-401, respectively. FIG. 25D depicts currents in the presence of varying concentrations of SN-418C and SN-401 normalized to the currents activated in hypotonic solution alone. Lines show the best fits to a logistic equation with IC50 of 265 nM and 4.0 μM for SN-418C and SN-401, respectively.

FIG. 26A-26B depict how SN-418C treatment dose-dependently impairs platelets activation. FIG. 26A depicts flow cytometry mean fluorescence intensity (MFI) of αIIbβ3 in washed platelets upon activation with thrombin or CRP agonists and dose-dependent inhibition by SN-418C. * p<0.05, ** p<0.001. FIG. 26B depicts flow cytometry mean fluorescence intensity (MFI) of P-selectin in washed platelets upon activation with thrombin or CRP agonists and dose-dependent inhibition by SN-418C. * p<0.05.

FIG. 27A-27B depict how SN-418C treatment inhibits agonist-stimulated platelet aggregation. FIG. 27A depicts washed platelets pretreated with different concentration of SN418C, and aggregation experiment was performed by aggregometer in presence of CRP. FIG. 27B depicts washed platelets pretreated with different concentration of SN418C, and aggregation experiment was performed by aggregometer in presence of thrombin agonist.

FIG. 28 depicts how SN418C inhibits thrombosis in FeCl₃-induced carotid injury. Carotid artery Time to total occlusion (TTO) is shown with vehicle (n=6) and 10 mg/kg i.p. SN-418C (n=7); p=0.06.

FIG. 29A-29C depicts how the human MK cell line, MEG01, had robust, SN-4XX-inhibited, swell-activated currents. FIG. 29A depicts current density over time in response to swelling with hypotonic solution (HYPO, 210 mOsm), and inhibition by 10 μM SN401 in WT MEG01 cells. FIG. 29B depicts the current-voltage relationship in WT MEG01 cells in response to HYPO and inhibition by 10 μM SN401. FIG. 29C depicts the current-voltage relationship in WT MEG01 cells in response to HYPO and inhibition by 3 μM SN418c.

### DETAILED DESCRIPTION

Various aspects of the present invention are directed to methods of preventing and/or treating thrombosis. Methods include methods for preventing and/or treating cardiovascular thrombosis in a patient in need of such therapy, comprising administering a therapeutically effective amount of an LRRC8 protein modulator to the patient. The method can comprise administering to the patient a therapeutically effective amount of an LRRC8 protein modulator to the patient so as to prevent and/or treat conditions caused or exacerbated by cardiovascular thrombosis including but not limited to myocardial ischemia, myocardial infarction, cerebrovascular transient ischemic attack, cerebrovascular ischemic stroke, or peripheral arterial occlusion. The method can comprise administering to the patient a therapeutically effective amount of an LRRC8 protein modulator to the patient so as to prevent and/or treat cardiovascular thrombosis, and conditions resulting from cardiovascular thrombosis, associated with atrial dysrhythmias including atrial flutter and atrial fibrillation. The method can comprise administering to the patient a therapeutically effective amount of an LRRC8 protein modulator to the patient so as to prevent and/or treat cardiovascular thrombosis associated with the acute or chronic use of medical devices exposed to intravascular blood including but not limited to intravascular diagnostic and treatment catheters, intravascular blood pumps, ventricular assist devices, intra-aortic balloon pumps, extracorporeal membrane oxygenation devices, prosthetic cardiac valves, and cardiac implantable electronic devices.

The disclosure is further directed to methods for preventing and/or treating hereditary or acquired defects in platelet function in a patient in need of such therapy, comprising administering a therapeutically effective amount of an LRRC8 protein modulator to the patient. The method can comprise administering to the patient a therapeutically effective amount of an LRRC8 protein modulator to the patient so as to normalize platelet function by increasing release or membrane expression of the contents of platelet alpha granules, dense granules, or other intracellular organelles contents including but not limited to platelet activating agents, clotting factors, and adhesion molecules. The method can comprise administering to the patient a therapeutically effective amount of an LRRC8 protein modulator to the patient so as to normalize platelet function by normalizing platelet volume.

The disclosure is further directed to methods for preventing and/or treating hereditary or acquired thrombocytosis in a patient in need of such therapy, comprising administering a therapeutically effective amount of an LRRC8 protein modulator to the patient.

The LRRC8 modulator can be DCPIB.

The administration of the compound can be sufficient to upregulate the expression of LRRC8 or expression of an LRRC8-associated protein. The administration of the compound can be sufficient to stabilize LRRC8 protein complexes or an LRRC8-associated protein. The administration of the compound can be sufficient to promote membrane trafficking and activity of LRRC8 protein complexes or an LRRC8-associated protein. The administration of the compound can be sufficient to augment LRRC8-mediated signaling or trafficking to the membrane of intra-cellular organelles including but not limited to lysosomal-related organelles.

In various embodiments, methods preventing and/or treating thrombosis in a subject in need thereof comprises administering to a subject a therapeutically effective amount of DCPIB (4-[2[butyl-6,7-dichloro-2-cyclopentyl-2,3-dihydro-1-oxo-1H-inden-5-yl)oxy]butanoic acid or a congener thereof.

In some embodiments, the method comprises administering to a subject a therapeutically effective amount of a compound selected from the group consisting of:

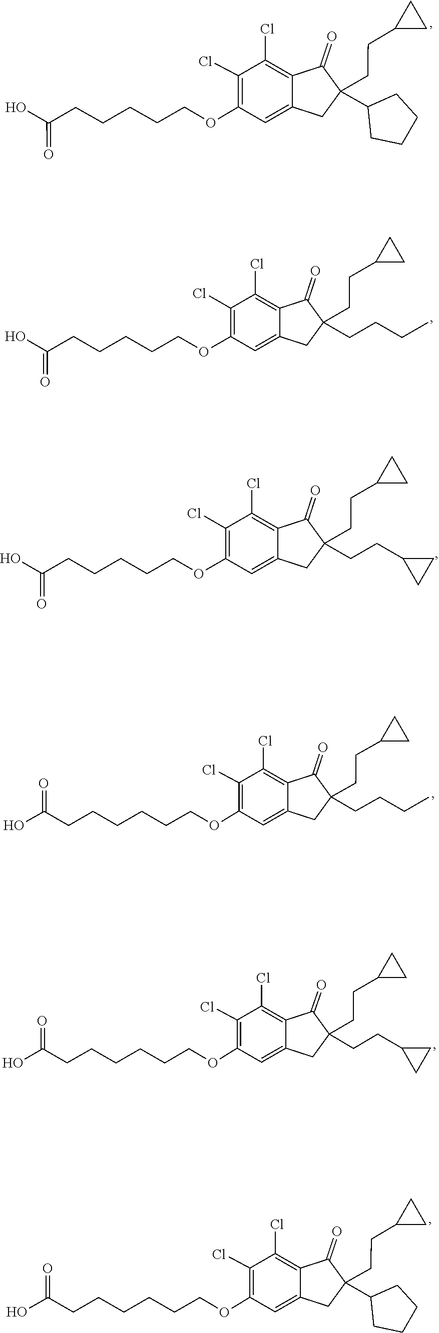 [SMILES: O=C(O)CCCCCOc1cc2c(c(Cl)c1Cl)C(=O)C(CCC1CC1)(C1CCCC1)C2 ; CCCCC1(CCC2CC2)Cc2cc(OCCCCCC(=O)O)c(Cl)c(Cl)c2C1=O ; O=C(O)CCCCCOc1cc2c(c(Cl)c1Cl)C(=O)C(CCC1CC1)(CCC1CC1)C2 ; CCCCC1(CCC2CC2)Cc2cc(OCCCCCCC(=O)O)c(Cl)c(Cl)c2C1=O ; O=C(O)CCCCCCOc1cc2c(c(Cl)c1Cl)C(=O)C(CCC1CC1)(CCC1CC1)C2 ; O=C(O)CCCCCCOc1cc2c(c(Cl)c1Cl)C(=O)C(CCC1CC1)(C1CCCC1)C2]

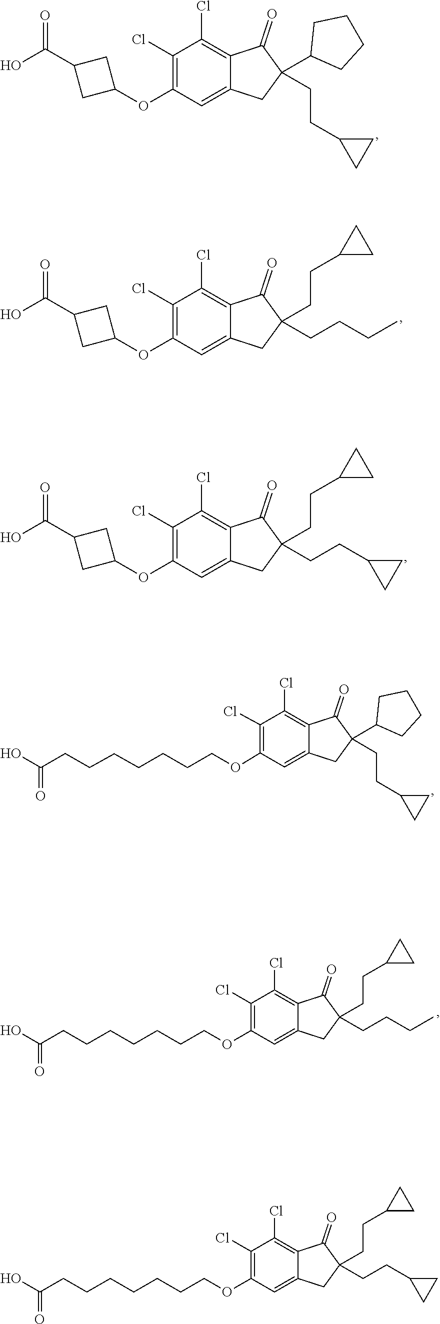 [SMILES: O=C(O)C1CC(Oc2cc3c(c(Cl)c2Cl)C(=O)C(CCC2CC2)(C2CCCC2)C3)C1 ; CCCCC1(CCC2CC2)Cc2cc(OC3CC(C(=O)O)C3)c(Cl)c(Cl)c2C1=O ; O=C(O)C1CC(Oc2cc3c(c(Cl)c2Cl)C(=O)C(CCC2CC2)(CCC2CC2)C3)C1 ; O=C(O)CCCCCCCOc1cc2c(c(Cl)c1Cl)C(=O)C(CCC1CC1)(C1CCCC1)C2 ; CCCCC1(CCC2CC2)Cc2cc(OCCCCCCCC(=O)O)c(Cl)c(Cl)c2C1=O ; O=C(O)CCCCCCCOc1cc2c(c(Cl)c1Cl)C(=O)C(CCC1CC1)(CCC1CC1)C2]

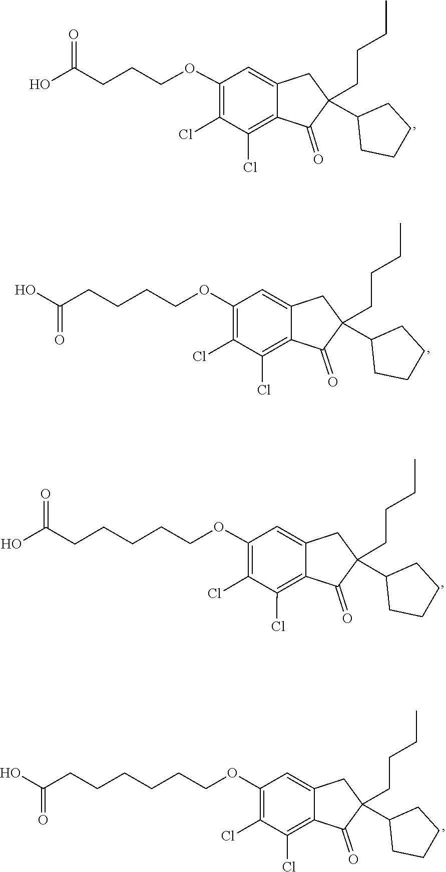 [SMILES: CCCCC1(C2CCCC2)Cc2cc(OCCCC(=O)O)c(Cl)c(Cl)c2C1=O ; CCCCC1(C2CCCC2)Cc2cc(OCCCCC(=O)O)c(Cl)c(Cl)c2C1=O ; CCCCC1(C2CCCC2)Cc2cc(OCCCCCC(=O)O)c(Cl)c(Cl)c2C1=O ; CCCCC1(C2CCCC2)Cc2cc(OCCCCCCC(=O)O)c(Cl)c(Cl)c2C1=O]

salts and geometric isomers thereof.

In various embodiments, the methods comprise administering to a subject a therapeutically effective amount of a compound of Formula (I), and salts and geometric isomers thereof:

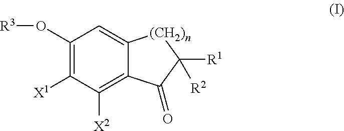

wherein R¹ and R² are each independently hydrogen, substituted or unsubstituted alkyl, substituted or unsubstituted alkenyl, substituted or unsubstituted alkoxy, substituted or unsubstituted aryl, substituted or unsubstituted heteroaryl;
R³ is —Y—C(O)R⁴, —Z—N(R⁵)(R⁶), or —Z-A;
A is selected from the group consisting of:

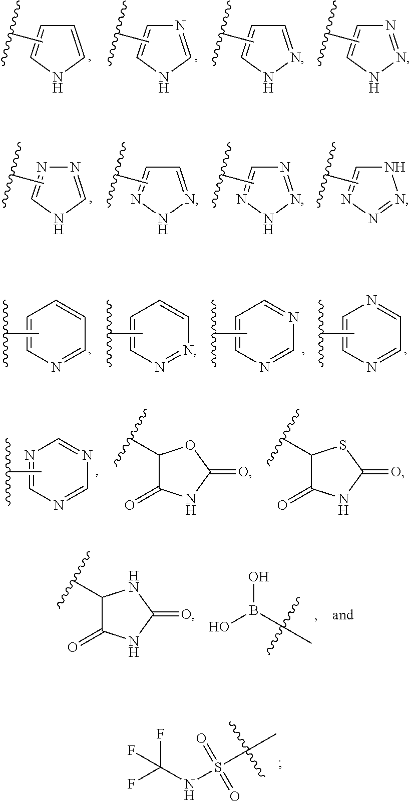

R⁴ is hydrogen, substituted or unsubstituted alkyl, —OR⁷, or —N(R⁸)(R⁹);
X¹ and X² are each independently substituted or unsubstituted alkyl, halo, —OR¹⁰, or —N(R¹¹)(R¹²); R⁵,
R⁶, R⁷, R⁸, R⁹, R¹⁰, R¹¹, and R¹² are each independently hydrogen or substituted or unsubstituted alkyl;
Y and Z are each independently a substituted or unsubstituted carbon-containing moiety having at least 2 carbon atoms; and n is 1 or 2.

In various embodiments, at least one of R¹ or R² is a substituted or unsubstituted linear or branched alkyl having at least 2 carbon atoms. In further embodiments, R¹ is hydrogen or a C1 to C6 alkyl. For example, in some embodiments, R¹ is butyl. In various embodiments, R² is cycloalkyl (e.g., cyclopentyl). In various embodiments, at least one of R¹ or R² is selected from the group consisting of:

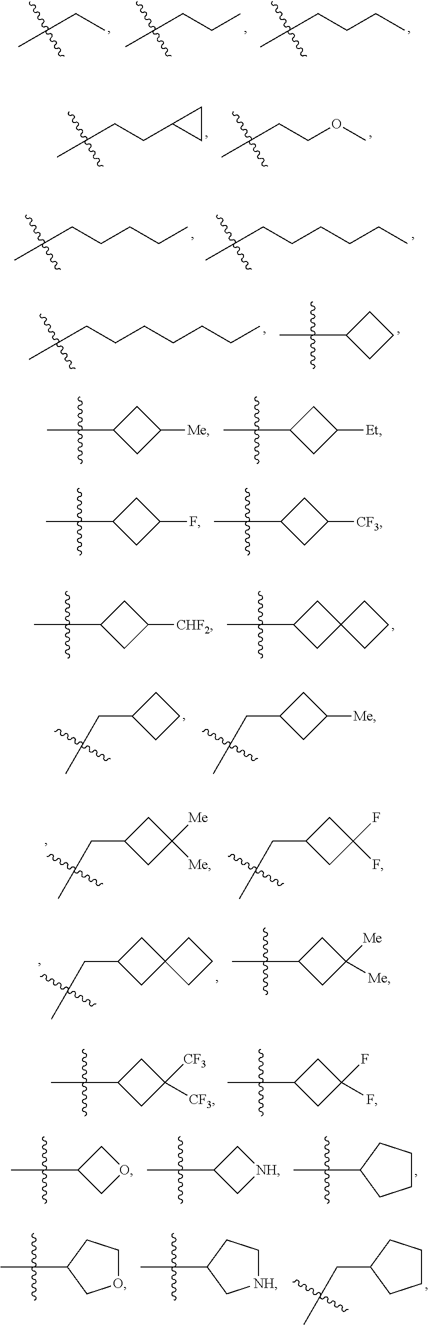

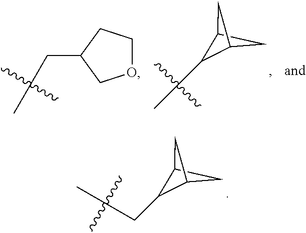 [SMILES: *CC1CCOC1 ; *C1C2CC1C2 ; *CC1C2CC1C2]

In various embodiments, R³ is —Y—C(O)R⁴. In various embodiments, R³ is —Z—N(R⁵)(R⁶). In various embodiments, R³ is —Z-A. In certain embodiments, R³ is selected from the group consisting of:

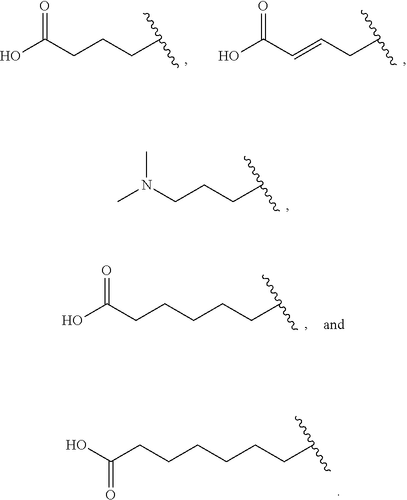

In certain embodiments R³ is selected from the group consisting of:

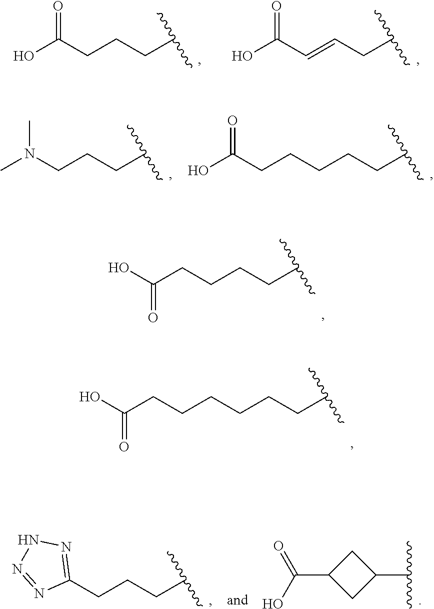

In various embodiments, A is selected from the group consisting of

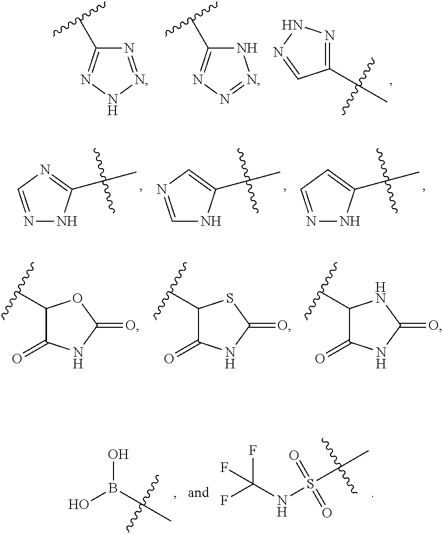

In various embodiments, R is —OR⁷ or —N(R⁸)(R⁹).

In various embodiments, X¹ and X² are each independently substituted or unsubstituted C₁ to C₆ alkyl or halo. In some embodiments, X¹ and X² are each independently C₁ to C₆ alkyl, fluoro, chloro, bromo, or iodo. In certain embodiments, X¹ and X² are each independently methyl, fluoro, or chloro.

In various embodiments, R₅, R⁶, R⁷, R⁸, R⁹, R¹⁰, R¹¹, and R¹² are each independently hydrogen or alkyl. For example, in some embodiments, R₅, R⁶, R⁷, R⁸, R⁹, R¹⁰, R¹¹, and R¹² are each independently hydrogen or a C₁ to C₃ alkyl.

In various embodiments, Y and Z are each independently substituted or unsubstituted alkylene having 2 to 10 carbons, substituted or unsubstituted alkenylene having from 2 to 10 carbons, or substituted or unsubstituted arylene. In some embodiments, Y and Z are each independently alkylene having 2 to 10 carbons, alkenylene having from 2 to 10 carbons, or phenylene. In certain embodiments, Y is an alkylene or an alkenylene having 3 to 8 or 3 to 7 carbons. In various embodiments, Y and Z are each independently cycloalkylene having 4 to 10 carbons. For example, Y can be an alkylene or any alkenylene having 4 carbons. In further embodiments, Z is an alkylene having 2 to 4 carbons. For example, Z can be an alkylene having 3 or 4 carbons. In certain embodiments, Y and Z are each independently selected from the group consisting of

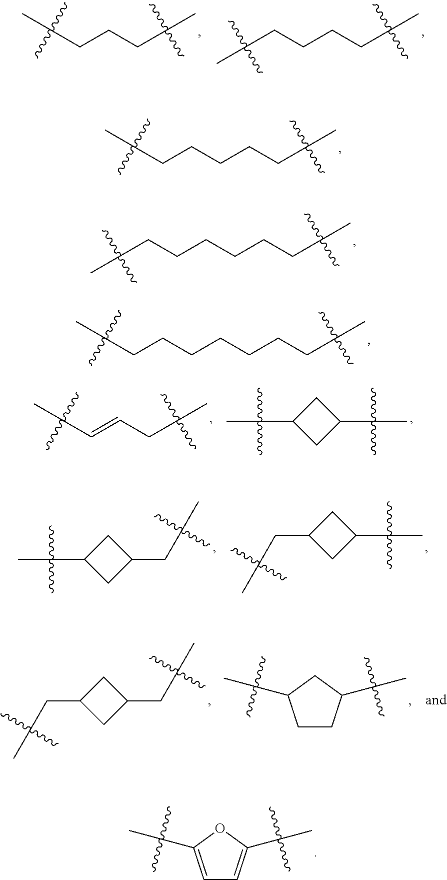

In various embodiments, when Y is an alkylene having 2 to 3 carbons then both X¹ and X² are each fluoro or each substituted or unsubstituted alkyl (e.g., methyl or ethyl). In some embodiments, Y is not an alkylene having 3 carbons. In certain embodiments, R⁷ is not hydrogen or a C1 to C6 alkyl. In some embodiments, X¹ and/or X² are not halo. In certain embodiments, X¹ and/or X² are not chloro. In some embodiments, R¹ and/or R² are not alkyl. In accordance with the embodiments, the compound of Formula (I) may be selected from the group consisting of:

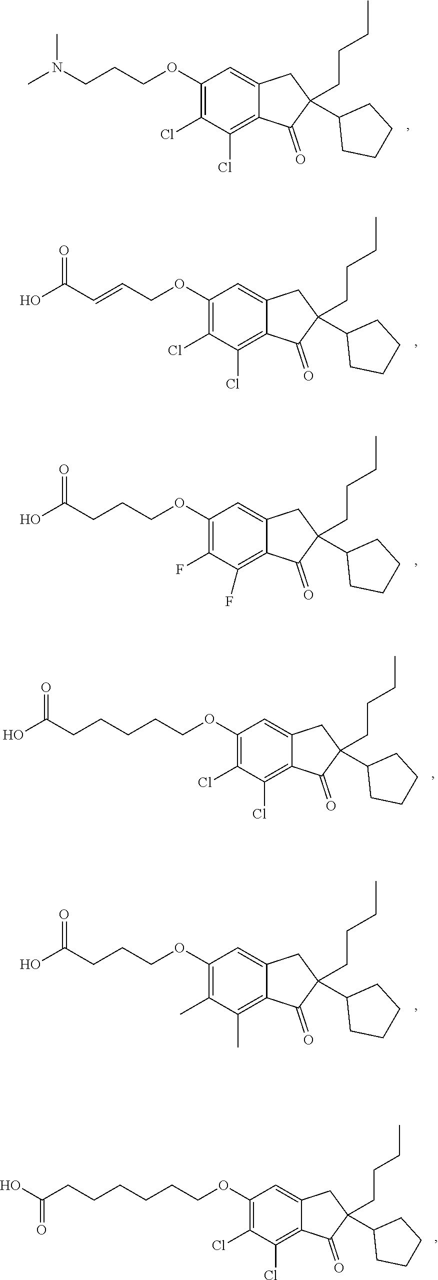 [SMILES: CCCCC1(C2CCCC2)Cc2cc(OCCCN(C)C)c(Cl)c(Cl)c2C1=O ; CCCCC1(C2CCCC2)Cc2cc(OC/C=C/C(=O)O)c(Cl)c(Cl)c2C1=O ; CCCCC1(C2CCCC2)Cc2cc(OCCCC(=O)O)c(F)c(F)c2C1=O ; CCCCC1(C2CCCC2)Cc2cc(OCCCCCC(=O)O)c(Cl)c(Cl)c2C1=O ; CCCCC1(C2CCCC2)Cc2cc(OCCCC(=O)O)c(C)c(C)c2C1=O ; CCCCC1(C2CCCC2)Cc2cc(OCCCCCCC(=O)O)c(Cl)c(Cl)c2C1=O]

and geometric isomers and salts thereof.

In certain the embodiments described herein, the compound to be administered is selected from the group consisting of:

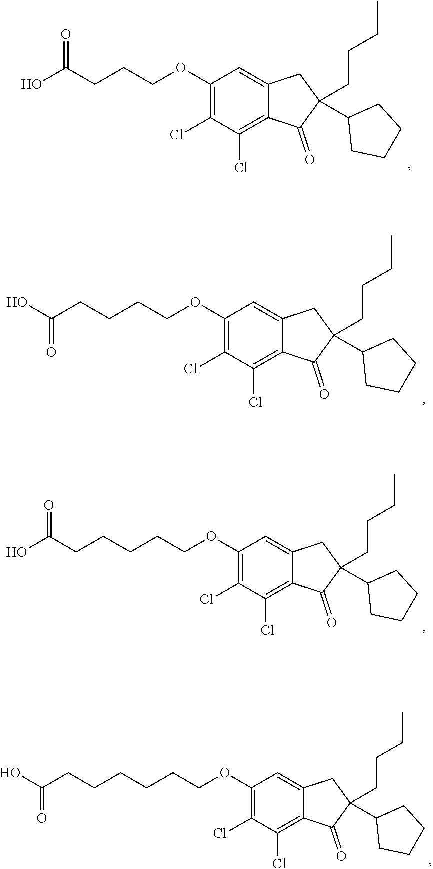 [SMILES: CCCCC1(C2CCCC2)Cc2cc(OCCCC(=O)O)c(Cl)c(Cl)c2C1=O ; CCCCC1(C2CCCC2)Cc2cc(OCCCCC(=O)O)c(Cl)c(Cl)c2C1=O ; CCCCC1(C2CCCC2)Cc2cc(OCCCCCC(=O)O)c(Cl)c(Cl)c2C1=O ; CCCCC1(C2CCCC2)Cc2cc(OCCCCCCC(=O)O)c(Cl)c(Cl)c2C1=O]

and geometric isomers and salts thereof.

The compounds to be administered can be selected from:

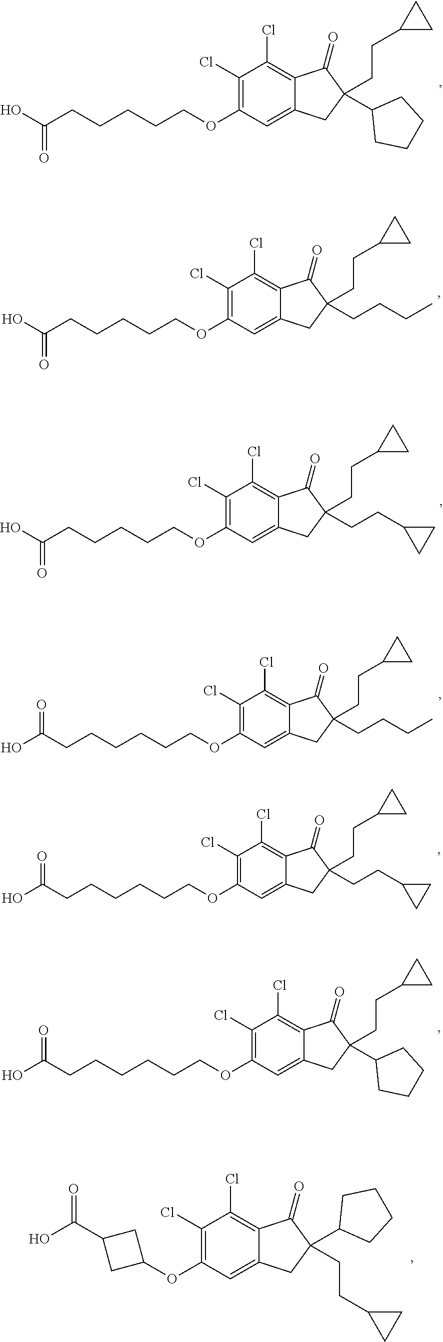

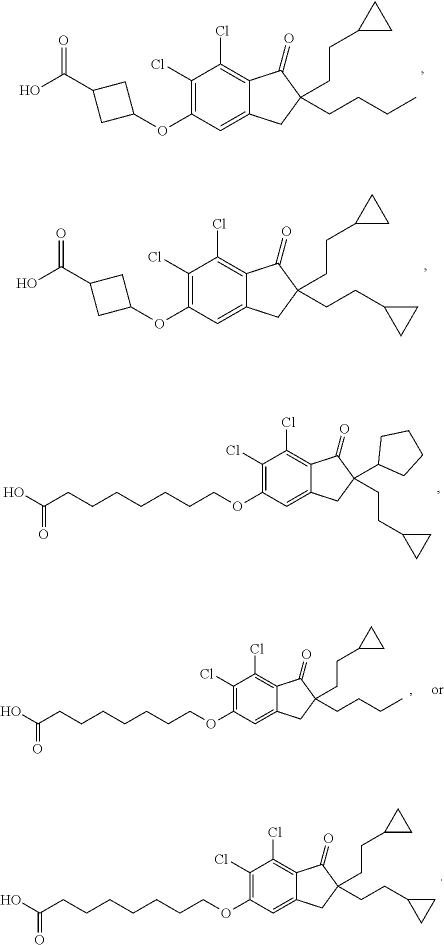 [SMILES: CCCCC1(CCC2CC2)Cc2cc(OC3CC(C(=O)O)C3)c(Cl)c(Cl)c2C1=O ; O=C(O)C1CC(Oc2cc3c(c(Cl)c2Cl)C(=O)C(CCC2CC2)(CCC2CC2)C3)C1 ; O=C(O)CCCCCCCOc1cc2c(c(Cl)c1Cl)C(=O)C(CCC1CC1)(C1CCCC1)C2 ; CCCCC1(CCC2CC2)Cc2cc(OCCCCCCCC(=O)O)c(Cl)c(Cl)c2C1=O ; O=C(O)CCCCCCCOc1cc2c(c(Cl)c1Cl)C(=O)C(CCC1CC1)(CCC1CC1)C2]

The disclosure is further directed to the compounds used in the methods described above. These compounds can modulate or inhibit an LRRC8 protein. The compound can have a higher potency at modulating or inhibiting a SWELL1 channel than an equivalent amount of DCPIB (4-[2[butyl-6,7-dichloro-2-cyclopentyl-2,3-dihydro-1-oxo-1H-inden-5-yl)oxy]butanoic acid).

In various embodiments, the methods of preventing or treating thrombosis include treating a disease in which thrombosis contributes to the disease state, the present invention relates to various methods of preventing or treating a disease in which cardiovascular thrombosis contributes to the disease state such as myocardial ischemia and infarction, stroke, transient cerebrovascular ischemia, peripheral arterial disease, thromboembolism associated with atrial fibrillation and flutter, and thromboembolism associated with the use of cardiovascular medical devices, as well as, disease related to platelet dysfunction including abnormal adhesion, aggregation, activation, or thrombosis.

Synthesis of the compound can proceed according to the following scheme:

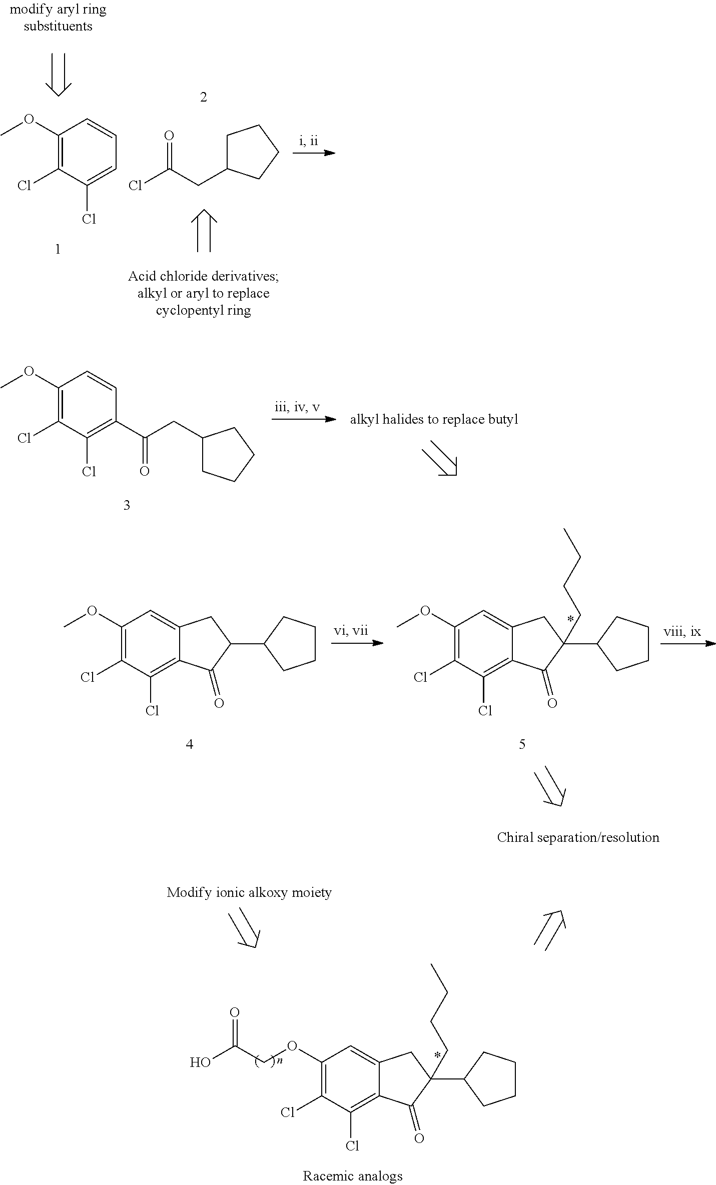

Modifications to the synthetic scheme above that can be made to synthesize a variety of compounds described herein are indicated by double arrows. Methods: i) AlCl₃, DCM, 5° C. to rt. ii) 12N HCl, iii) 1) Paraformaldehyde, dimethylamine, acetic acid, 85° C. iv) DMF, 85° C., v) H₂SO₄. vi) KOtBu, butyl iodide. vii) pyridine-HCl, 195° C. viii) BrCH₂CO₂Et, K₂CO₃, DMF, 60° C. ix) ION NaOH.

In accordance with the various methods of the present invention, a pharmaceutical composition comprising a compound of Formula (I) is administered to the subject in need thereof. The pharmaceutical composition can be administered by routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. In various embodiments, administration is selected from the group consisting of oral, intranasal, intraperitoneal, intravenous, intramuscular, rectal, and transdermal.

The determination of a therapeutically effective dose for any one or more of the compounds described herein is within the capability of those skilled in the art. A therapeutically effective dose refers to that amount of active ingredient which provides the desired result. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Typically, the normal dosage amount of the compound can vary from about 0.05 to about 100 mg per kg body weight depending upon the route of administration. Guidance as to particular dosages and methods of delivery is provided in the literature and generally available to practitioners in the art. It will generally be administered so that a daily oral dose in the range, for example, from about 0.1 mg to about 75 mg, from about 0.5 mg to about 50 mg, or from about 1 mg to about 25 mg per kg body weight is given. The active ingredient can be administered in a single dose per day, or alternatively, in divided doses (e.g., twice per day, three time a day, four times a day, etc.). In general, lower doses can be administered when a parenteral route is employed. Thus, for example, for intravenous administration, a dose in the range, for example, from about 0.05 mg to about 30 mg, from about 0.1 mg to about 25 mg, or from about 0.1 mg to about 20 mg per kg body weight can be used.

A pharmaceutical composition for oral administration can be formulated using pharmaceutically acceptable carriers known in the art in dosages suitable for oral administration. Such carriers enable the pharmaceutical compositions to be formulated as tablets, pills, dragees, capsules, liquids, gels, syrups, slurries, suspensions, and the like, for ingestion by the subject. In certain embodiments, the composition is formulated for parenteral administration. Further details on techniques for formulation and administration can be found in the latest edition of REMINGTON'S PHARMACEUTICAL SCIENCES (Mack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

In addition to the active ingredients (e.g., the compound of Formula (I)), the pharmaceutical composition can contain suitable pharmaceutically acceptable carriers comprising excipients and auxiliaries that facilitate processing of the active compounds into preparations which can be used pharmaceutically. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Unless otherwise indicated, the alkyl, alkenyl, and alkynyl groups described herein preferably contains from 1 to 20 carbon atoms in the principal chain. They may be straight or branched chain or cyclic (e.g., cycloalkyls). Alkenyl groups can contain saturated or unsaturated carbon chains so long as at least one carbon-carbon double bond is present. Alkynyl groups can contain saturated or unsaturated carbon chains so long as at least one carbon-carbon triple bond is present. Unless otherwise indicated, the alkoxy groups described herein contain saturated or unsaturated, branched or unbranched carbon chains having from 1 to 20 carbon atoms in the principal chain.

Unless otherwise indicated herein, the term “aryl” refers to monocyclic, bicyclic or tricyclic aromatic groups containing from 6 to 14 ring carbon atoms and including, for example, phenyl. The term “heteroaryl” refers to monocyclic, bicyclic or tricyclic aromatic groups having 5 to 14 ring atoms and containing carbon atoms and at least 1, 2 or 3 oxygen, nitrogen or sulfur heteroatoms.

Having described the invention in detail, it will be apparent that modifications and variations are possible without departing from the scope of the invention defined in the appended claims.

### EXAMPLES

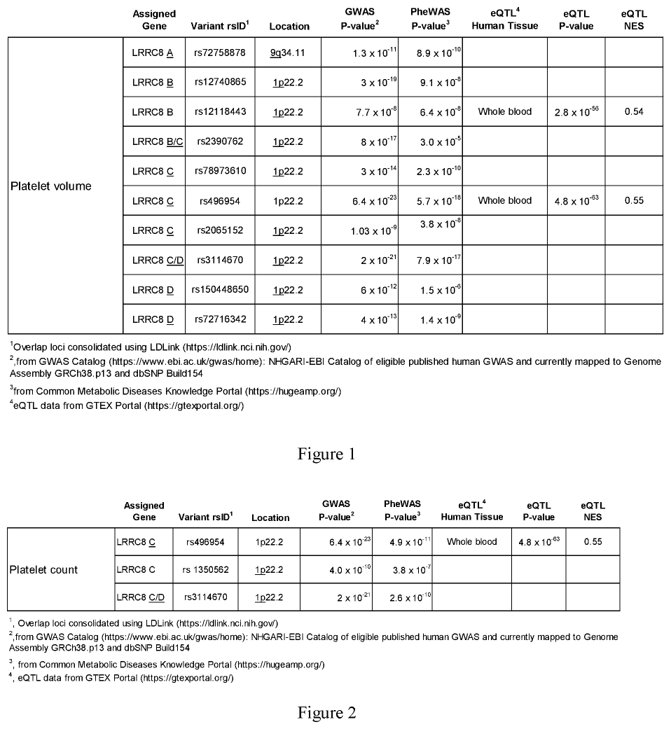

### Example 1: Genome-Wide Association Studies (GWAS) Indicate LRRC8 Proteins Regulate Human Platelet Volume (FIG. 1)

A search of the NHGARI-EBI Catalog of eligible published human genome-wide association studies (accessed 9 Feb. 2022) was conducted. That catalog of data is currently mapped to Genome Assembly GRCh38.p13 and dbSNP Build154. The NHGARI-EBI Catalog of published human GWAS studies listed 13 SNP variants assigned to the five LRRC8 genes associated with 6 metabolic, inflammatory, and CV traits with P-values <10⁻⁸. Each of the five LRRC8 genes contained at least one of these 13 SNPs (accessed 2 Sep. 2022). It was found that GWAS in the database identified associations between platelet volume and 6 SNP variants assigned to three genes on two chromosomes (LRRC8 A (2×10⁻¹⁰) on 9q34.11 and LRRC8 C (5×10⁻²³ & 3×10⁻¹⁴) and LRRC8 D (4×10⁻¹³ & 6×10⁻¹²) on 1p22.2).

### Example 2: Phenome-Wide Association Studies (PheWAS) Indicate LRRC8 Proteins Regulate Human Platelet Volume (FIG. 1)

After identifying 6 SNPs associated with human platelet volume in the NHGARI-EBI Catalog of published human GWAS studies, the Common Metabolic Diseases Knowledge Portal (accessed 3 Mar. 2022) and the PheWAS-IEU OpenGWAS Project catalog (PheWAS Catalog) were searched for PheWAS associations between platelet volume and the 13 SNP variants with P-values <10⁻⁵ (accessed 2 Sep. 2022). PheWAS identified an association between these SNP variants and platelet volume (8.9×10⁻¹⁰, 2.3×10⁻¹⁰, 5.7×10⁻¹⁸, 7.9×10⁻¹⁷, 1.5×10⁻⁶ and 1.4×10⁻⁹), as well as, 4 additional SNP variants assigned to LRRC8 B, the interval of overlap between LRRC8 B and C, and LRRC8 C that were initially identified because they are associated with Blood Monocyte Count or Diastolic Blood Pressure in the GWAS catalog. In all, the Platelet Volume phenotype is associated with 10 SNPs assigned to 4 LRRC8 genes (LRRC8 A-D) on 2 chromosomes (1p22.2 & 9q34.11).

### Example 3: EQTL Studies Indicate LRRC8 Proteins Regulate Human Platelet Volume (FIG. 1)

The GTEX Portal database expression quantitative trait loci (eQTL) database (accessed 5 Mar. 2022) was searched, and significant associations were found between levels of mRNA transcripts for LRRC8B (P=2.8×10⁻⁵⁶) and LRRC 8C (P=4.8×10⁻⁶³) in whole blood and SNPs assigned to those genes that were associated with platelet volume in the NHGARI-EBI Catalog of published human GWAS studies.

### Example 4: Phenome-Wide Association Studies (PheWAS) Indicate LRRC8 Proteins Regulate Human Platelet Count (FIG. 2)

The Common Metabolic Diseases Knowledge Portal (accessed 3 Mar. 2022) was searched for PheWAS associations between platelet count and the 13 SNP variants with P-values <10⁻⁵ (accessed 2 Sep. 2022). PheWAS identified an association between 3 SNP variants and platelet count (4.9×10⁻¹¹, 2.6×10⁻¹⁰, and 3.8×10⁻⁷). In all, the platelet count phenotype is associated with 3 SNPs assigned to 2 LRRC8 genes (LRRC8 C-D) on 1 chromosome (lp22.2).

### Example 5: EQTL Studies Indicate LRRC8 Proteins Regulate Human Platelet Count (FIG. 2)

The GTEX Portal database expression quantitative trait loci (eQTL) database (accessed 5 Mar. 2022) was searched, and significant associations were found between levels of mRNA transcripts for LRRC 8C (P=4.8×10⁻⁶³) in whole blood and a SNP assigned to LRRC 8C that was associated with platelet count in the Common Metabolic Diseases Knowledge Portal (accessed 3 Mar. 2022) or the PheWAS-IEU OpenGWAS Project catalog (PheWAS Catalog).

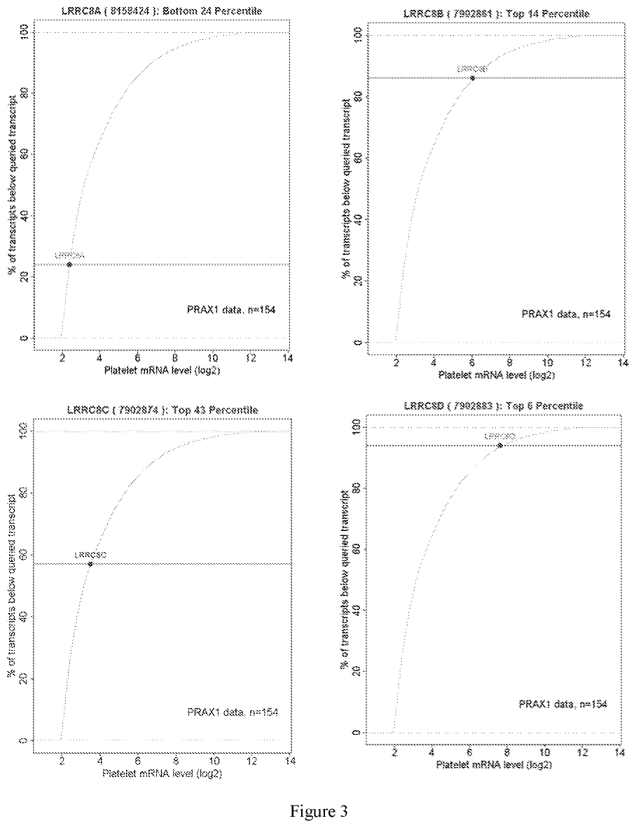

### Example 6: LRRC8A-D mRNA Transcripts are Expressed in Human Platelets at High Levels Compared to Other mRNA Transcripts (FIG. 3)

The Plateletomics Interactive Results Tool version 1.0 was used to search the Plateletomics database (accessed 5 Mar. 2022), and it was found that mRNA transcripts for LRRC 8A-8D are expressed in human platelets and that they are in the top 76^{th}, 14^{th}, 43^{rd}, and top 5^{th} percentile, respectively, of all mRNA transcripts detected in the platelets of 154 patients. This indicates mRNA transcripts for LRRC 8A-D are highly expressed in platelets compared to other mRNA transcripts.

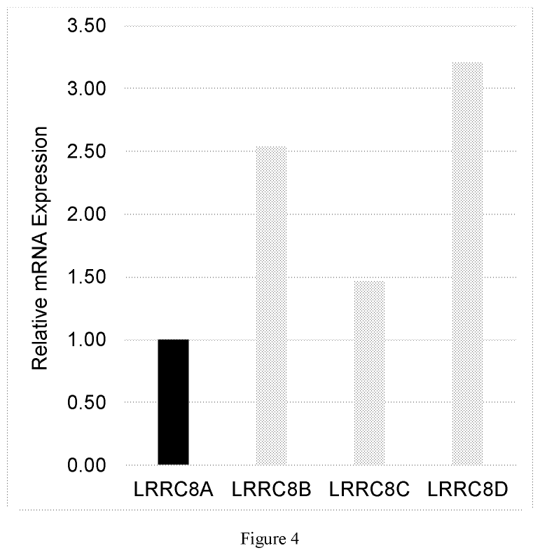

### Example 7: Human Platelets Contain mRNA Transcripts for LRRC 8A, LRRC 8B, LRRC 8C, and LRRC8D in Proportions of 1 to 2.5 to 1.5 to 3.0, Respectively (FIG. 4)

LRRC 8A-E transcripts were searched for in the database in Supplementary Table S2A from the 2014 publications by Simon, L. M. et al. (Blood 123, e37-45 (2014)), and it was found that LRRCA, LRRC 8B, LRRC 8C, and LRRC 8D mRNA transcripts are expressed in human platelets, and that LRRC 8B, LRRC8C, and LRRC8D are expressed at approximately 2.5 fold, 1.5 fold, and 3-fold the levels of LRRC 8A mRNA, respectively.

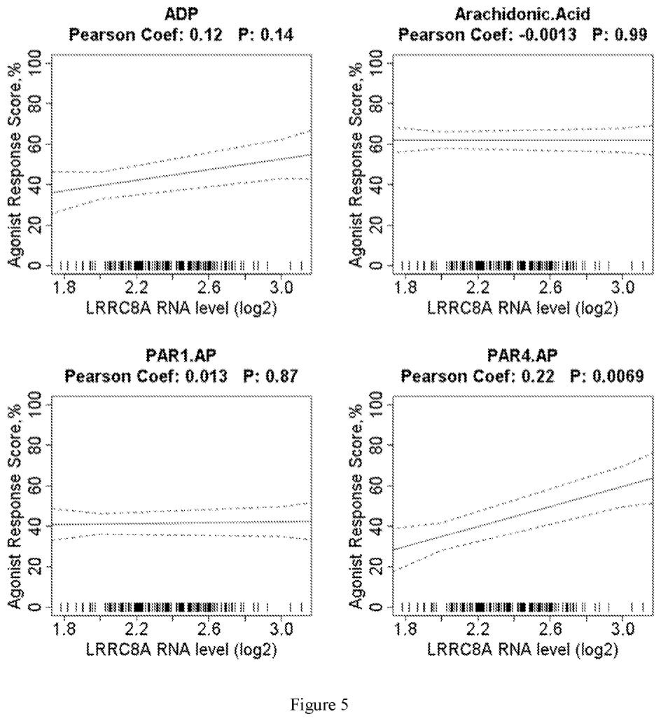

### Example 8: The Degree of Human Platelet Aggregation Stimulated by Protease-Activated Receptor 4 (PAR4) Agonist is Proportional to LRRC8A Expression (FIG. 5)

The Plateletomics Interactive Results Tool version 1.0 was used to search the Plateletomics database (accessed 5 Mar. 2022), and data was found that demonstrated that the intensity of human platelet aggregation stimulated by protease-activated receptor 4-activating peptide (PAR4 AP), an agonist for the platelet PAR4 thrombin receptor, is significantly associated with the amount of LRRC8A mRNA expressed in human platelets.

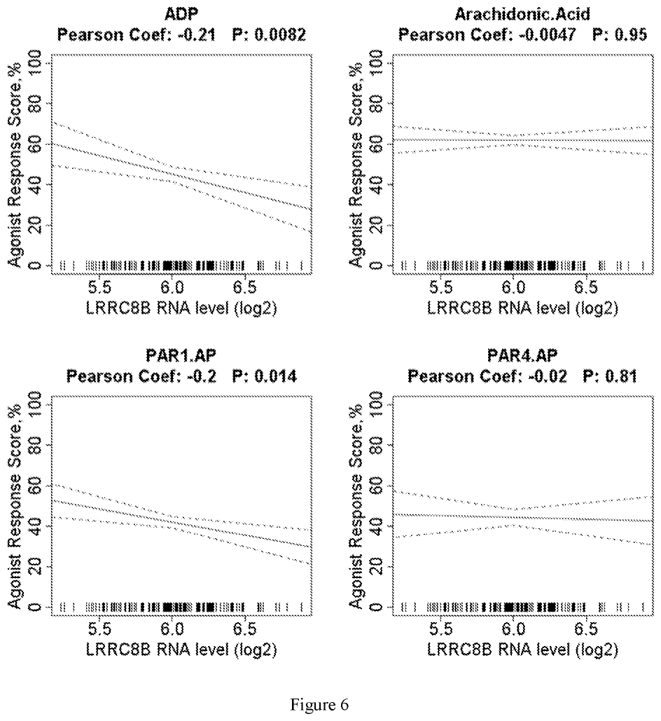

### Example 9: The Degree of Human Platelet Aggregation Stimulated by Adenosine Diphosphate (ADP) and Protease-Activated Receptor 1 (PAR1) Agonist are Inversely Proportional to LRRC8B Expression. (FIG. 6)

The Plateletomics Interactive Results Tool version 1.0 was used to search the Plateletomics database (accessed 5 Mar. 2022), and data was found that demonstrated that the intensity of human platelet aggregation stimulated by adenosine diphosphate (ADP), an agonist for the platelet P2Y12 and other ADP receptors, and by protease-activated receptor 1-activatin peptide (PAR1 AP), an agonist for the platelet PAR1 thrombin receptors, are significantly inversely associated with the amount of LRRC8B mRNA expressed in human platelets.

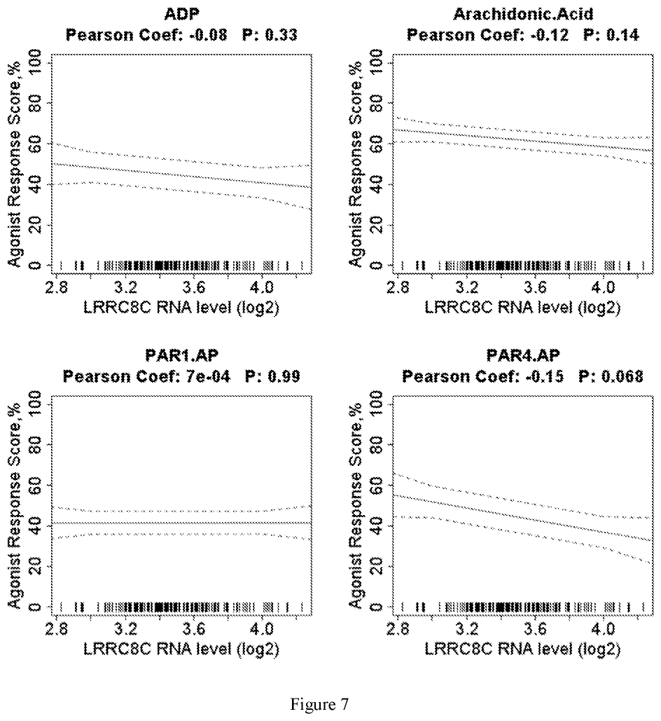

### Example 10: The Degree of Human Platelet Aggregation Stimulated by Protease-Activated Receptor 4 (PAR4) Agonist is Inversely Proportional to LRRC8C Expression (FIG. 7)

The Plateletomics Interactive Results Tool version 1.0 was used to search the Plateletomics database (accessed 5 Mar. 2022), and data was found that demonstrated that the intensity of human platelet aggregation stimulated by protease-activated receptor 4-activating peptide (PAR4 AP), an agonist for the platelet PAR4 thrombin receptor, is associated inversely associated with the amount of LRRC8C mRNA expressed in human platelets.

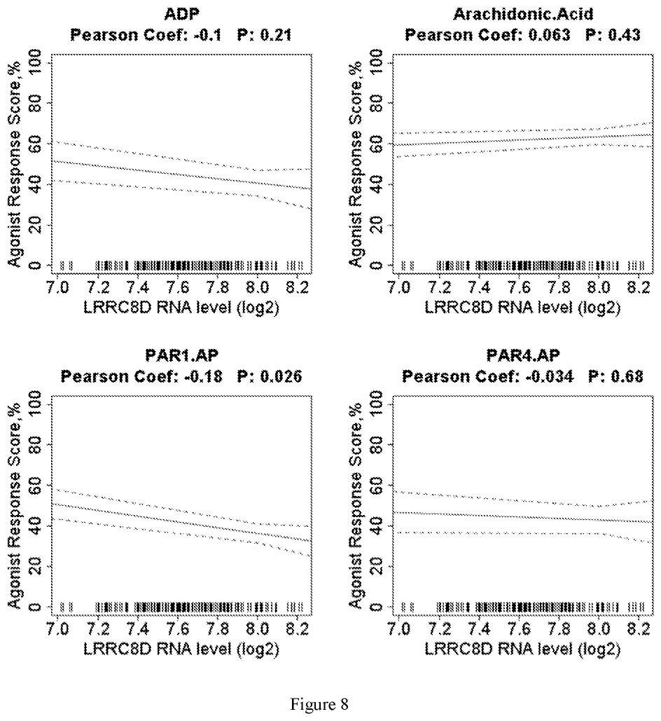

### Example 11: The Degree of Human Platelet Aggregation Stimulated by Protease-Activated Receptor 1 (PAR1) Agonist is Inversely Proportional to LRRC8D Expression (FIG. 8)

The Plateletomics Interactive Results Tool version 1.0 was used to search the Plateletomics database (accessed 5 Mar. 2022), and data was found that demonstrated that the intensity of human platelet aggregation stimulated by protease-activated receptor 1-activating peptide (PAR1 AP), an agonist for the platelet PAR1 thrombin receptor, is associated inversely associated with the amount of LRRC8D mRNA expressed in human platelets.

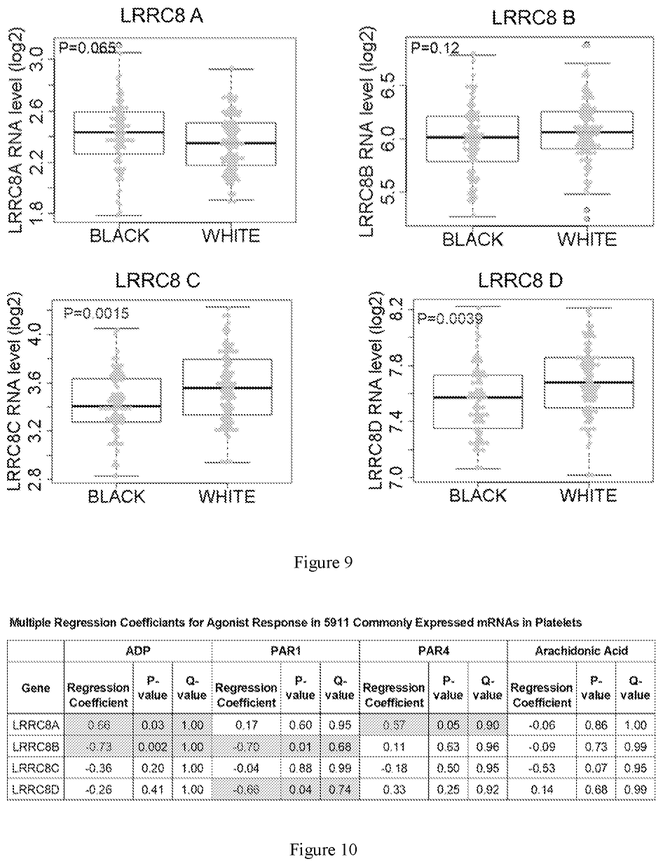

### Example 12: There are Racial Differences in LRRC8A-D Expression in Human Platelets (FIG. 9)

The Plateletomics Interactive Results Tool version 1.0 was used to search the Plateletomics database (accessed 5 Mar. 2022), and data was found that demonstrated that LRRC8C and LRRC8D mRNA expression is significantly lower in people identified as Black compared to people identified as White. LRRC8A and LRRC8B gene expression are also lower in people identified as Black compared to people identified as White, but did not exceed the threshold for significance in this dataset.

### Example 13: Multiple Regression Analyses Demonstrate LRRC8A, LRRC8B, and LRRC8D Play Significant Roles in Human Platelet Aggregation in Response to ADP, PAR1 Agonist Peptide, and PAR4 Agonist Peptide (FIG. 10)

Regression coefficients for LRRC 8A-D in multiple regression analyses for platelet aggregation response in 5911 commonly expressed mRNAs in human platelets from 154 human subjects from the 2014 Supplementary Table S3 of Simon, L. M. et al. (Blood 123, e37-45 (2014)), and the corresponding values for tests of significance demonstrate that the degree of ADP-induced platelet aggregation is significantly associated with the level of LRRC8A mRNA and the inverse of the level of LRRC8B mRNA; the degree of PAR1-induced platelet aggregation is significantly associated with the inverse of the level of LRRC8B and LRRC8D mRNA; and the degree of PAR4-induced platelet aggregation is associated with the level of LRRC8D mRNA. The regression specification dependent variable was the degree of platelet aggregation in response to one of four platelet agonists (ADP, PAR1 AP, PAR 2AP, and arachidonic acid), and the independent variables were age, race, gender, platelet number, and BMI, and the levels of 5,911 commonly expressed mRNAs.

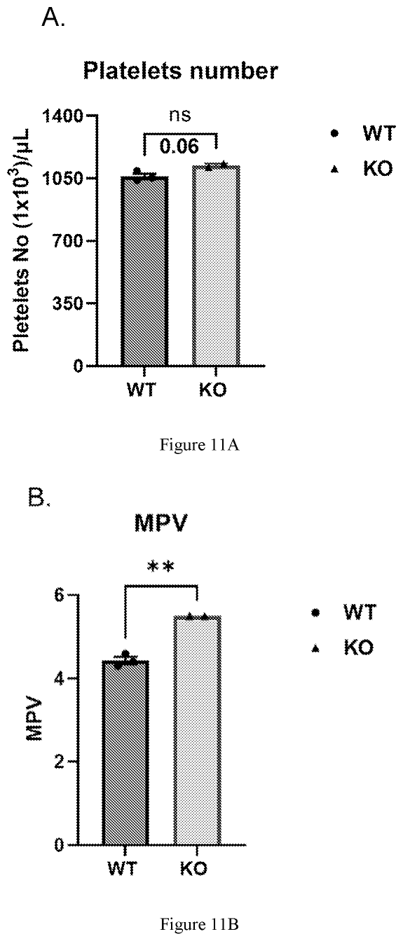

### Example 14: LRRC8A in Platelets Regulates Platelet Volume In Vivo (FIG. 11)

Whole blood was collected from the inferior vena cava of 20-week old WT mice and mice lacking platelet LRRC8A into heparin anticoagulant (15 USP units/mL), and a whole blood CBC was run on an Element HT5 veterinary hematology analyzer (Heska, Loveland) which demonstrated the mean platelet volume (MPV) of platelets lacking LRRC8A was significantly greater than the MPV of WT platelets (P<0.01; unpaired, two-sided t-test). The platelet count was also increased in the mice with platelets lacking LRRC8A compared to WT mice, but the difference did not cross the threshold for significance (P=00.06).

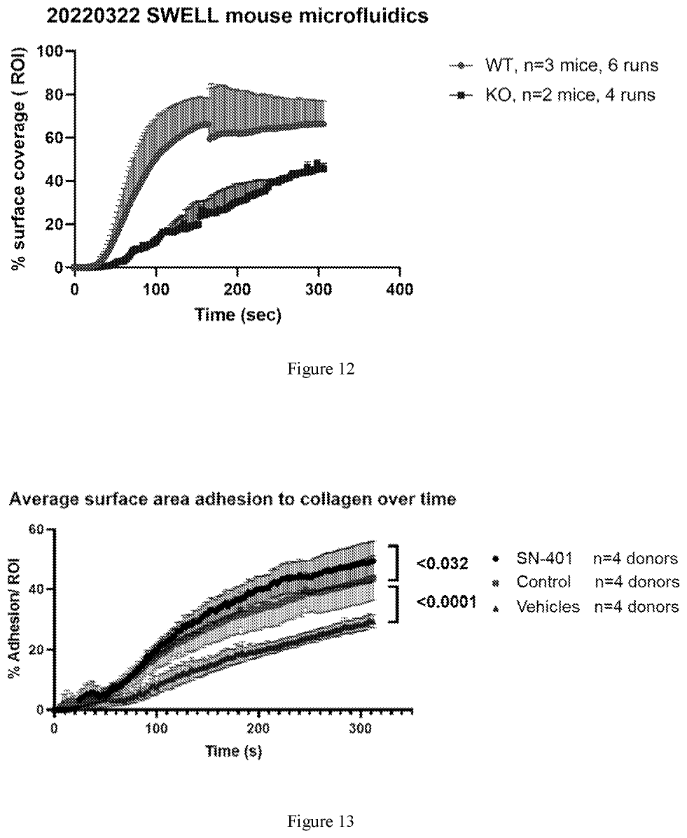

### Example 15: LRRC8A is Required for Normal Platelet Adhesion of Human Platelets to Collagen (FIG. 12)

Whole blood was collected from the inferior vena cava of 20-week old WT mice and mice lacking platelet LRRC8A into heparin anticoagulant. The heparinized whole blood was fluorescently labeled with DiOC6 (3,39-dihexyloxacarbocyanine iodide). The whole blood was perfused through a polydimethysiloxane (PDMS) microfluidic device plasma bonded to a glass slide patterned with bovine type 1 collagen at a constant upstream pressure for 5 minutes followed by PBS for 5 minutes. Platelet accumulation was captured using an Olympus IX83 inverted microscope and the surface area coverage of platelets quantified using ImageJ. It was found that absence of LRRC8A significantly reduced platelet adhesion (P<0.0001; two-tailed paired t-test).

### Example 16: Inhibition of LRRC8 with SN-401 (DCPIB) Significantly Inhibited Adhesion of Human Platelets to Collagen (FIG. 13)

Human blood from 4 donors was collected into heparin anticoagulant, pooled and incubated with the fluorescently label DiOC6 (3,39-dihexyloxacarbocyanine iodide; Invitrogen) and either 10 μM SN-401 (DCPIB) or vehicle (DMSO) for 10 minutes. The whole blood was perfused through a polydimethysiloxane (PDMS) microfluidic device plasma bonded to a glass slide patterned with bovine type 1 collagen at a constant upstream pressure for 5 minutes followed by PBS for 5 minutes. Platelet accumulation was captured using an Olympus IX83 inverted microscope and the surface area coverage of platelets quantified using ImageJ. It was found that SN-401 significantly reduced adhesion of human platelets to collagen (vehicle v. SN-401; P<0.0001; ordinary one-way ANOVA with Turkeys multiple comparison tests).

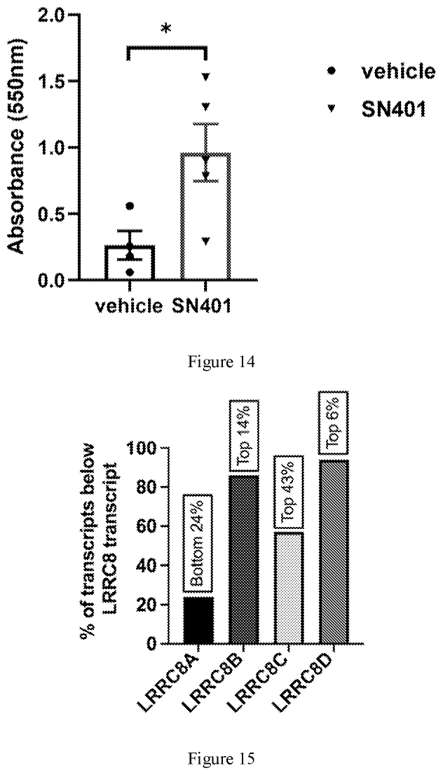

### Example 17: Inhibition of LRRC8 with SN-401 (DCPIB) Significantly Inhibited Thrombosis In Vivo (FIG. 14)

To determine if acute inhibition of LRRC8 channel activity affected platelet function in vivo, vehicle or the known LRRC8 channel inhibitor SN-401 (DCPIB) was administered to 25 to 30-week old C57BL6 mice by IP injection, and in vivo thrombosis was quantified using an assay for tail wound thrombosis. Specifically, 20 minutes following IP injection with vehicle or 10 mg/kg SN-401, mouse tails were amputated 5 mm from the tail tip, placed in normal saline at 37° C. for 15 minutes, and the relative blood loss during that period was quantified by estimating the hemoglobin concentration in the normal saline by measuring light absorbance at 550 nm. It was found that the mean amount of tail blood loss in mice receiving SN-401 was 4-fold the blood loss in mice receiving vehicle (P<0.05; unpaired two-tailed t-test). This demonstrated pharmacologic inhibition of LRRC8 inhibited thrombosis in vivo.

### Example 18: Functional Studies (FIGS. 15-29C)

As detailed above, 4 of 5 LRRC8 sub-unit genes, LRRC8A-D, were shown to be associated with mean platelet volume (MPV) in genome-wide association studies (GWAS) and phenome-wide association studies (PheWAS), implicating LRRC8 channels in platelet function and thrombosis in humans. These findings are supported by human platelet mRNA expression and platelet activation data in the Plateletomics database, which reveal 1) LRRC8A-D are expressed in platelets (FIG. 15) and 2) transcript levels of LRRC8A, 8B, and 8D are significantly correlated to platelet activation by known soluble platelet agonists (P<0.007 to P<0.03). Notably, LRRC8E is not detected in human platelets and also was not associated with platelet characteristics in the GWAS/PheWAS, which is consistent with LRRC8A, B, C, and D being genuine associations.

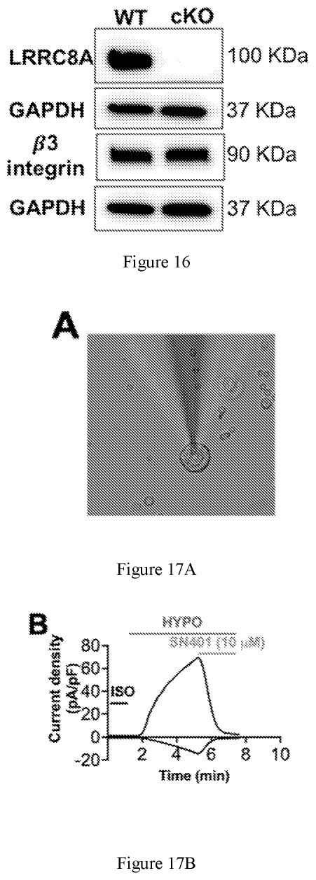

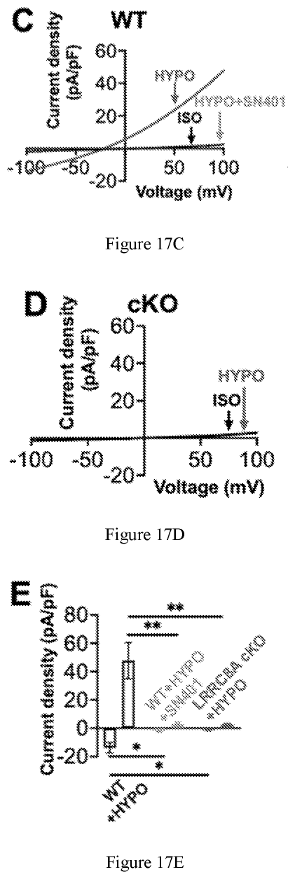

To validate the biology implied by the human genetic studies, megakaryocyte (MK)-specific LRRC8A conditional knockout (cKO) mice were generated by crossing our LRRC8A^{fl/fl} mouse (SWELL1^{fl/fl})³⁵⁻³¹ with the platelet factor4-driven Cre recombinase mouse (Pf4-Cre) to generate LRRC8A^{fl/fl;Pf4-cre+/-} (cKO) and littermate control LRRC8A^{fl/fl} mice (WT). Complete deletion of LRRC8A protein from cKO mouse platelets was confirmed while maintaining expression of the platelet marker b3 integrin (FIG. 16). To confirm that LRRC8 proteins form functional VRAC/I_{CI,SWELL} channels in platelets, I_{CI,SWELL} was measured in freshly isolated murine MK using the perforated patch configuration of the patch-clamp technique (FIG. 17A). Robust I_{CI,SWELL} currents were observed in response to hypotonic swelling in WT MK that was fully inhibited upon application of 10 mM of tool compound SN-401, with virtually no appreciable background currents (FIG. 17B-17D). This hypotonically-activated, swell-activated current is entirely abolished in cKO MKs (FIG. 17C-17D), confirming the SN-401-inhibited current in murine MKs to be entirely LRRC8A mediated.

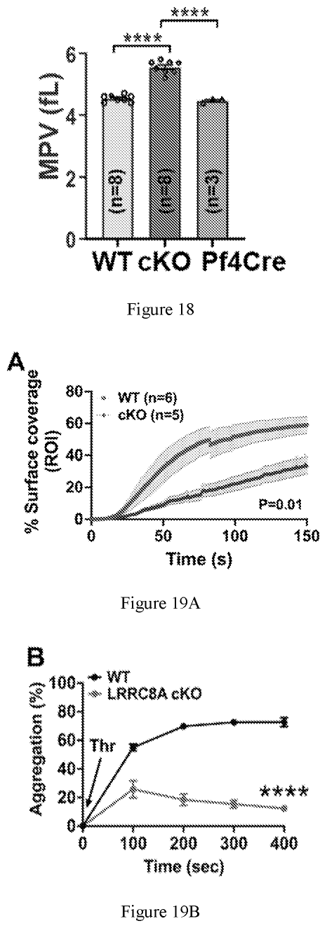

Having confirmed ablation of LRRC8A protein and LRRC8A-mediated function in cKO platelets, platelet volume and number were next measured. Consistent with the increase in MPV associated with the SNP assigned to LRRC8A in GWAS/PheWAS studies, platelet-targeted deletion of LRRC8A also increases MPV compared to both WT and platelet-targeted heterozygous controls (FIG. 18), without increasing platelet count. Thus, deletion of LRRC8A in mouse platelets causes an increase in MPV, consistent with the variant human SNP allele located in the 5′-untranslated exonic region of LRRC8A identified in GWAS/PheWAS studies.

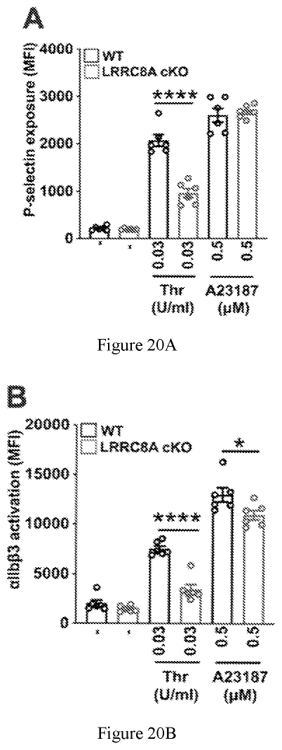

Next, the functional consequences of LRRC8A deletion in platelets were examined. Platelet adhesion, mediated primarily by collagen receptor GPVI, alpha- and dense-granule secretion, and aggregation mediated by activated aIIbb3 integrin fibrinogen receptors are three platelet functions essential for normal thrombus formation. LRRC8A depletion markedly impairs platelet adhesion to a fibrillar collagen-coated surface under shear conditions (FIG. 19A) and thrombin-stimulated aggregation (FIG. 19B). Furthermore, LRRC8A deletion reduced thrombin-stimulated platelet activation as assessed by P-selectin exposure, a measure of alpha-granule release (FIG. 20A) and αIIbb3 integrin activation (FIG. 20B). However, LRRC8A-null platelets are still responsive to the Ca²⁺ ionophore A23187, suggesting that LRRC8 regulates platelet signaling pathways upstream of intracellular Ca²⁺. In summary, these data show LRRC8 channel signaling is required for normal platelet volume, adhesion, granule release, and aggregation.

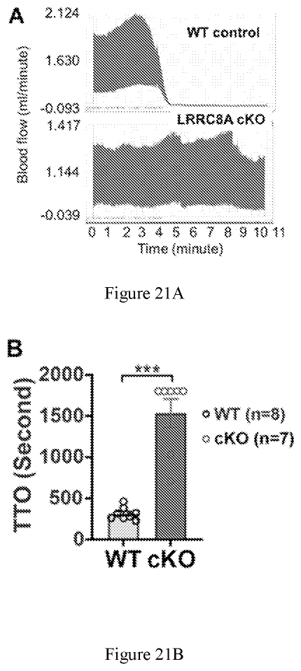

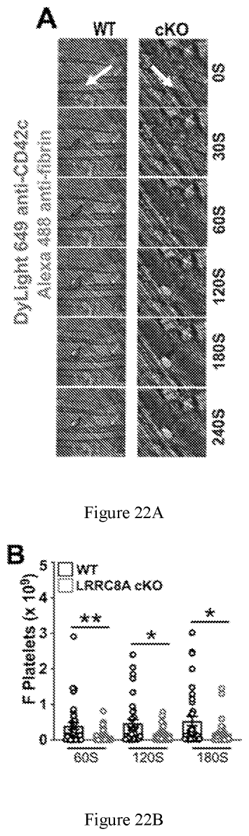

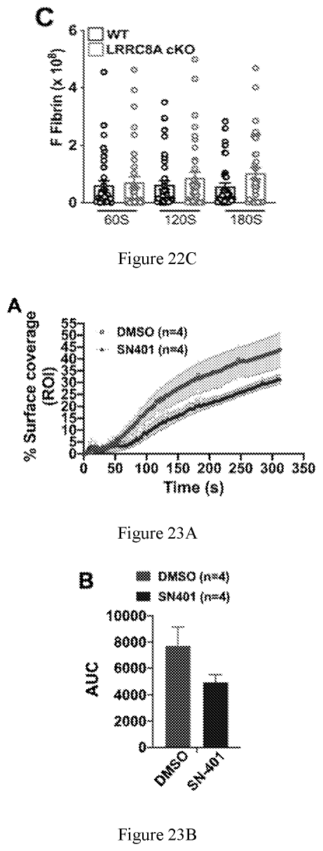

Since in vivo thrombosis integrates a number of additional factors beyond platelet aggregation and adhesion observed in vitro, we examined arterial thrombosis in WT and cKO mice using two different in vivo thrombosis models: (1) FeCl₃-induced carotid arterial injury (FIG. 21A-21B); and (2) laser-induced cremaster arteriolar injury (FIGS. 22A, 22B, and 22C). Remarkably, marked prolongation of the time to occlusion (ITO) of FeCl₃ injured carotid arteries of cKO mice were observed as compared to WT mice (FIG. 21A-21B). Notably, as shown in FIG. 21B, 5/7 cKO mice had TO greater than the 30 min (1800 s) time limit of the experiment, and therefore the mean TTO underestimates the true TTO because those values are censored at 30 minutes. In the laser-induced cremaster arteriolar injury model, MK-specific deletion of LRRC8A also significantly reduced platelet thrombus formation compared to WT mice (FIGS. 22A, 22B, and 22C), while fibrin generation was not affected in cKO mice (FIGS. 22A, 22B, and 22C), consistent with a platelet-specific defect in LRRC8 signaling.

Moreover, preserved fibrin generation in cKO mice suggests that pharmacological inhibition may have the potential to selectively impair platelet thrombus formation, while preserving hemostasis associated with intact fibrin generation suggesting LRRC8 channels may be a drug target less prone to major bleeding, while still being an effective anti-thrombotic agent. Collectively these data, spanning human genetics to murine platelet-targeted loss of function models, rigorously validate platelet LRRC8 channels as an innovative drug target for an antiplatelet agent with a previously unknown mechanism of action. Moreover, platelet LRRC8 inhibitors are being developed as anti-thrombotics for use in both non-diabetic and diabetic patients.

Furthermore, it was shown that SN-4XX compounds inhibit platelet activity.

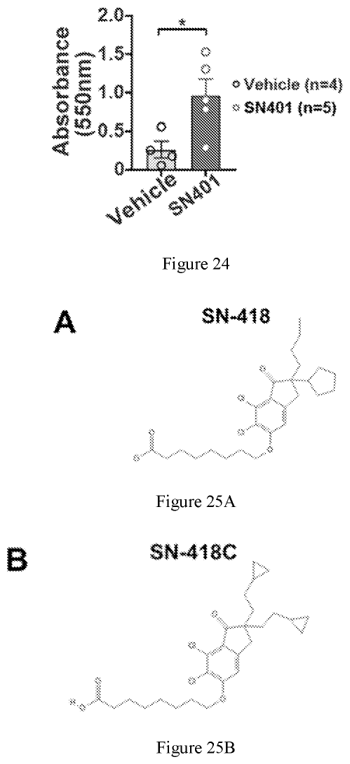

Patch-clamp studies with tool compound SN-401 reveal robust inhibition of LRRC8 channel activity in murine MK (FIG. 17A-17E), suggesting SN-401 may also inhibit platelet activity if LRRC8 channel activity is required for platelet activation. Preliminary platelet adhesion studies performed on human blood (4 different donors, performed on 4 separate days) using microfluidic devices demonstrated SN-401 significantly inhibited platelet adhesion (FIG. 23A-23B). Consistent with this, i.p. injection of 10 mg/kg SN-401 in mice revealed an increase in tail bleeding in vivo (FIG. 24). These data provide proof of concept that LRRC8 channel inhibition with SN-4XX compounds have antiplatelet, and anti-thrombotic activity.

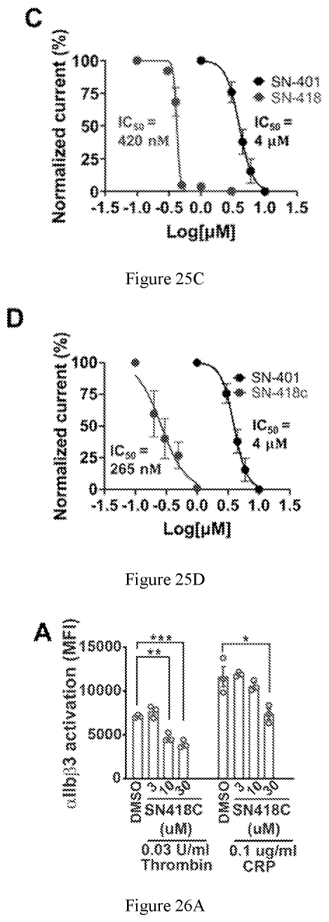

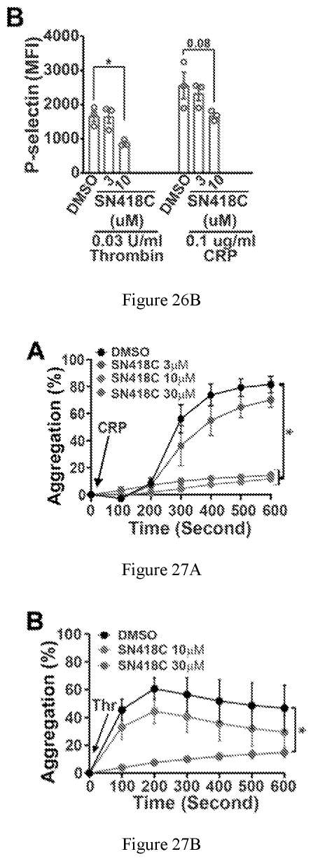

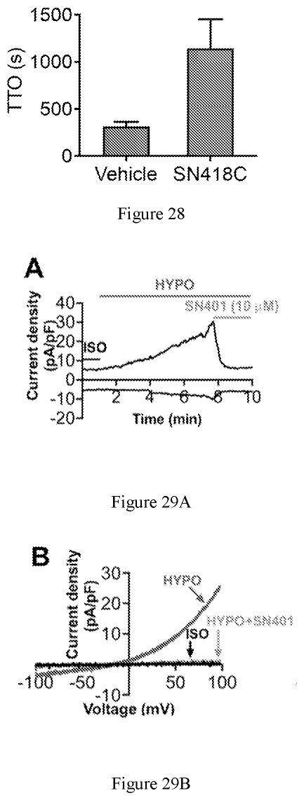

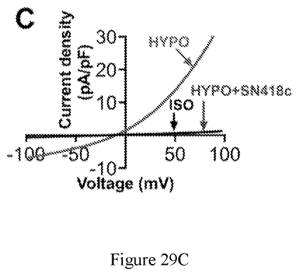

A SAR-based approach has been successfully applied to develop more potent LRRC8 channel inhibitors. Examples of these are SN-418 and SN-418C (FIG. 25A&25B), which include modification in the alkyl chain+/−cyclopropyl group to increase LRRC8 channel inhibition ˜10-fold over SN-401 (FIG. 25C&25D). SN-418C was then tested for antiplatelet activity in vitro. Significant inhibition of thrombin-induced platelet activation was observed as assessed by aIIbb3 integrin activation (FIG. 26A) and P-selectin exposure (FIG. 26B). Consistent with these findings, SN-418C also robustly inhibited platelet aggregation in response to collagen-related peptide (CRP) and thrombin in a dose-dependent manner (FIG. 27A-27B). In fact, this level of platelet aggregation inhibition was comparable to the observations in LRRC8A null platelets (FIG. 19B). Finally, i.p. injection of 10 mg/kg SN-418C for 4 days and then 20 min prior to FeCl₃ carotid arterial injury showed that SN-418C markedly increases the time to occlusion (ITO, FIG. 28) 3.7-fold over vehicle treated mice. Similar to what was observed with LRRC8A cKO mice (FIG. 21A-21B), SN-418C treatment resulted in 4/7 mice with TO greater than the 30 min standard time limit of the experiment, indicating that the observed increase in iTO is underestimated at this 10 mg/kg dose SN-418 dose. As shown in FIG. 29A, FIG. 29B, and FIG. 29C, MEG01 cells also have robust I_{CI,SWELL} current in response to hypotonic swelling, and these are completely inhibited by SN-401 and SN-418C.

Overall, these data indicate that SN-40X compounds have anti-platelet activity and may be used as anti-thrombotics. This provides a proof of concept for an anti-platelet agent that functions via a previously unknown mechanism of action, for the treatment of myocardial infarction, stroke, coronary arteries disease, peripheral artery disease, and other thrombotic diseases and complications.

All documents cited herein are incorporated by reference. While certain embodiments of invention are described, and many details have been set forth for purposes of illustration, certain of the details can be varied without departing from the basic principles of the invention. The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms 20 (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order 25 unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

What is claimed is:

1. A method for preventing and/or treating cardiovascular thrombosis in a patient in need of such therapy, comprising administering a therapeutically effective amount of an LRRC8 protein modulator to the patient.

2. The method of claim 1, wherein the method comprises administering to the patient a therapeutically effective amount of an LRRC8 protein modulator to the patient so as to prevent and/or treat conditions caused or exacerbated by cardiovascular thrombosis including but not limited to myocardial ischemia, myocardial infarction, cerebrovascular transient ischemic attack, cerebrovascular ischemic stroke, or peripheral arterial occlusion.

3. The method of claim 1, wherein the method comprises administering to the patient a therapeutically effective amount of an LRRC8 protein modulator to the patient so as to prevent and/or treat cardiovascular thrombosis, and conditions resulting from cardiovascular thrombosis, associated with atrial dysrhythmias including atrial flutter and atrial fibrillation.

4. The method of claim 1, wherein the method comprises administering to the patient a therapeutically effective amount of an LRRC8 protein modulator to the patient so as to prevent and/or treat cardiovascular thrombosis associated with the acute or chronic use of medical devices exposed to intravascular blood including but not limited to intravascular diagnostic and treatment catheters, intravascular blood pumps, ventricular assist devices, intra-aortic balloon pumps, extracorporeal membrane oxygenation devices, prosthetic cardiac valves, and cardiac implantable electronic devices.

5. A method for preventing and/or treating hereditary or acquired defects in platelet function in a patient in need of such therapy, comprising administering a therapeutically effective amount of an LRRC8 protein modulator to the patient.

6. The method of claim 5, wherein the method comprises administering to the patient a therapeutically effective amount of an LRRC8 protein modulator to the patient so as to normalize platelet function by increasing release or membrane expression of the contents of platelet alpha granules, dense granules, or other intracellular organelles contents including but not limited to platelet activating agents, clotting factors, and adhesion molecules.

7. The method of claim 5, wherein the method comprises administering to the patient a therapeutically effective amount of an LRRC8 protein modulator to the patient so as to normalize platelet function by normalizing platelet volume.

8. A method for preventing and/or treating hereditary or acquired thrombocytosis in a patient in need of such therapy, comprising administering a therapeutically effective amount of an LRRC8 protein modulator to the patient

9. The method of any one of claims 1 to 8, wherein the LRRC8 modulator is DCPIB.

10. The method of any one of the claims 1 to 9, wherein the LRRC8 modulator is a compound of Formula (I), or a geometric isomer or salt thereof: 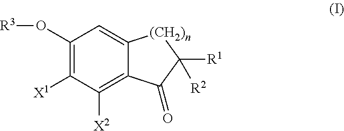
wherein:
R¹ and R² are each independently hydrogen, substituted or unsubstituted alkyl, substituted or unsubstituted alkenyl, substituted or unsubstituted alkoxy, substituted or unsubstituted aryl, substituted or unsubstituted heteroaryl;
R³ is —Y—C(O)R⁴, —Z—N(R⁵)(R⁶), or —Z-A;
A is selected from the group consisting of: 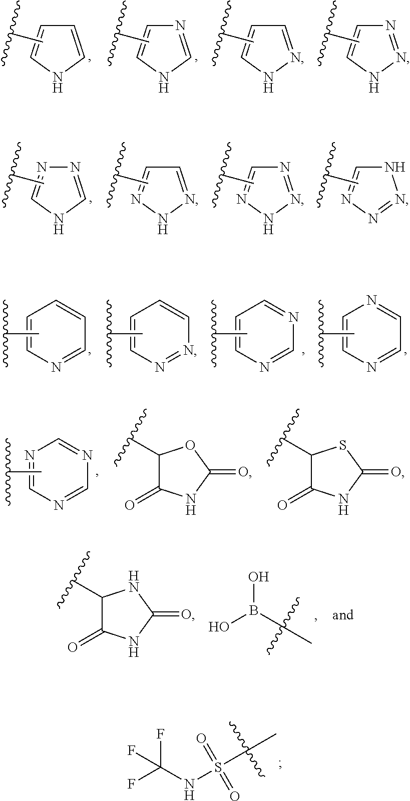
R⁴ is hydrogen, substituted or unsubstituted alkyl, —OR⁷, or —N(R⁸)R⁹);
X¹ and X² are each independently substituted or unsubstituted alkyl, halo, —OR¹⁰, or —N(R¹¹)(R¹²);
R⁵, R⁶, R⁷, R⁸, R⁹, R¹⁰, R¹¹, and R¹² are each independently hydrogen or substituted or unsubstituted alkyl;
Y and Z are each independently a substituted or unsubstituted carbon-containing moiety having at least 2 carbon atoms; and
n is 1 or 2.

11. The method of claim 10, wherein at least one of R¹ or R² is a substituted or unsubstituted linear or branched alkyl having at least 2 carbon atoms.

12. The method from claim 10 or 11, wherein at least one of R¹ or R² is selected from the group consisting of:

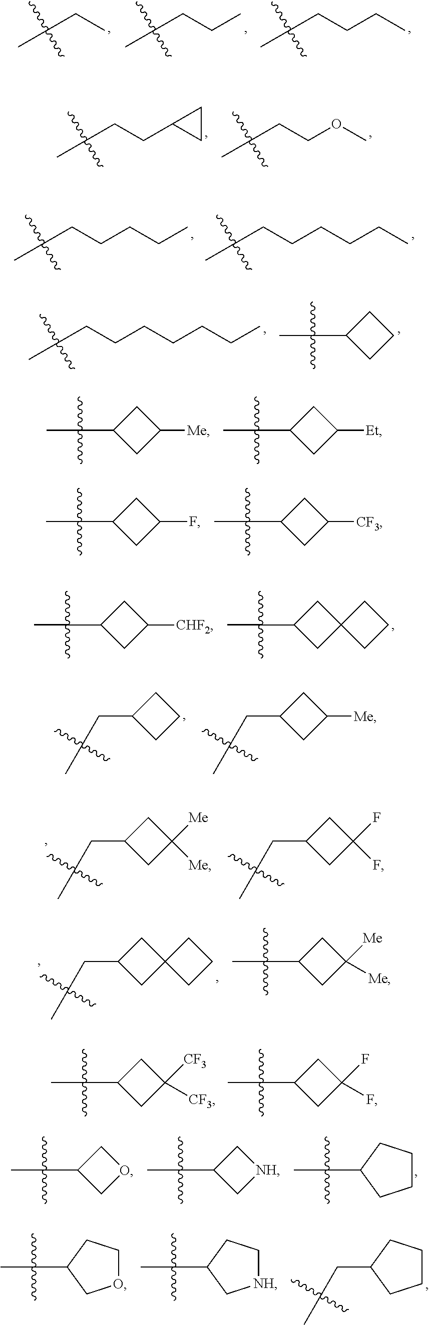

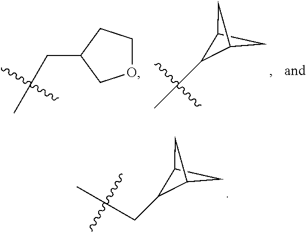 [SMILES: *CC1CCOC1 ; *C1C2CC1C2 ; *CC1C2CC1C2]

13. The method of claim 10 to 12, wherein R¹ is hydrogen or a C1 to C6 alkyl.

14. The method of any one of claims 10 to 13, wherein R¹ is butyl.

15. The method of any one of claims 10 to 14, wherein R² is cycloalkyl.

16. The method of any one of claims 10 to 15, wherein R² is cyclopentyl.

17. The method of any one of claims 10 to 16, wherein R³ is —Y—C(O)R⁴.

18. The method of any one of claims 10 to 17, wherein R⁴ is —OR⁷ or —N(R⁸)R⁹).

19. The method of any one of claims 10 to 18, wherein R³ is —Z—N(R⁵)(R⁶).

20. The method of any one of claims 10 to 19, wherein R³ is —Z-A.

21. The method of any one of claims 10 to 20, wherein A is selected from the group consisting of

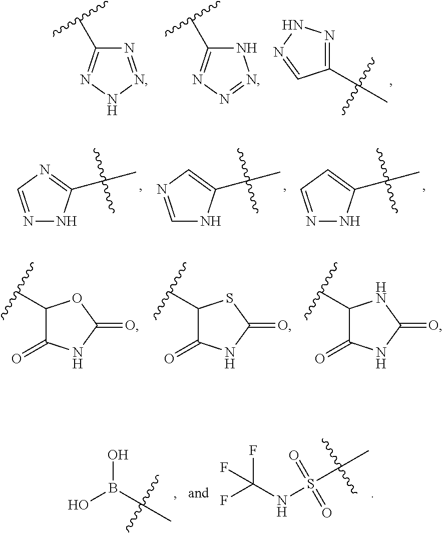

22. The method of any one of claims 10 to 21, wherein Y and Z are each independently substituted or unsubstituted alkylene having 2 to 10 carbons, substituted or unsubstituted alkenylene having from 2 to 10 carbons, or substituted or unsubstituted arylene.

23. The method of any one of claims 10 to 22, wherein Y and Z are each independently alkylene having 2 to 10 carbons, alkenylene having from 2 to 10 carbons, or phenylene.

24. The method of any one of claims 10 to 23, wherein Y and Z are each independently cycloalkylene having 4 to 10 carbons.

25. The method of any one of claims 10 to 24, wherein Y is an alkylene or an alkenylene having 3 to 8 carbons or 3 to 7 carbons.

26. The method of any one of claims 10 to 25, wherein Y is an alkylene or any alkenylene having 4 carbons.

27. The method of any one of claims 10 to 26, wherein Z is an alkylene having 2 to 4 carbons.

28. The method of any one of claims 10 to 27, wherein Z is an alkylene having 3 or 4 carbons.

29. The method of any one of the claims 10 to 28, wherein Y and Z are each independently selected from the group consisting of

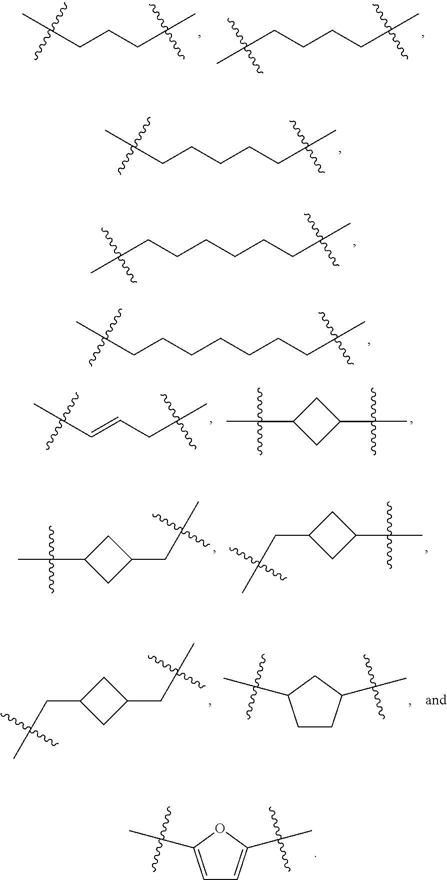

30. The method of any one of the claims 10 to 29, wherein when Y is an alkyl having 2 to 3 carbons then both X¹ and X² are each fluoro or each substituted or unsubstituted alkyl.

31. The method of any one of the claims 10 to 30, wherein R³ is selected from the group consisting of:

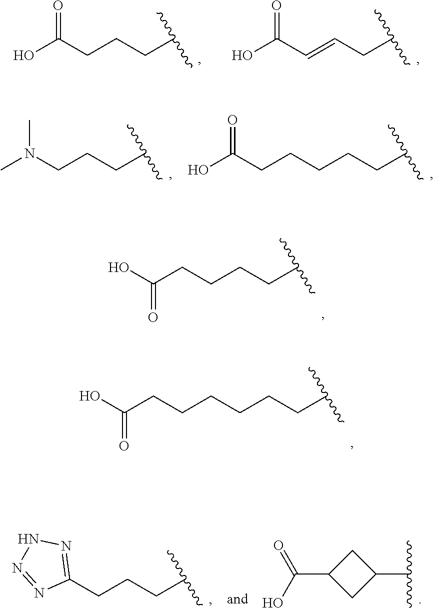

32. The method of any one of claims 10 to 31, wherein X¹ and X² are each independently substituted or unsubstituted C1 to C6 alkyl or halo.

33. The method of any one of claims 10 to 32, wherein X¹ and X² are each independently C1 to C6 alkyl, fluoro, chloro, bromo, or iodo.

34. The method of any one of claims 10 to 33, wherein X¹ and X² are each independently methyl, fluoro, or chloro.

35. The method of any one of claims 10 to 34, wherein R⁵, R⁶, R⁷, R⁸, R⁹, R¹⁰, R¹¹, and R¹² are each independently hydrogen or alkyl.

36. The method of any one of claims 10 to 35, wherein R⁵, R⁶, R⁷, R⁸, R⁹, R¹⁰, R¹¹, and R¹² are each independently hydrogen or a C1 to C3 alkyl.

37. The method of any one of claims 10 to 36 selected from the group consisting of:

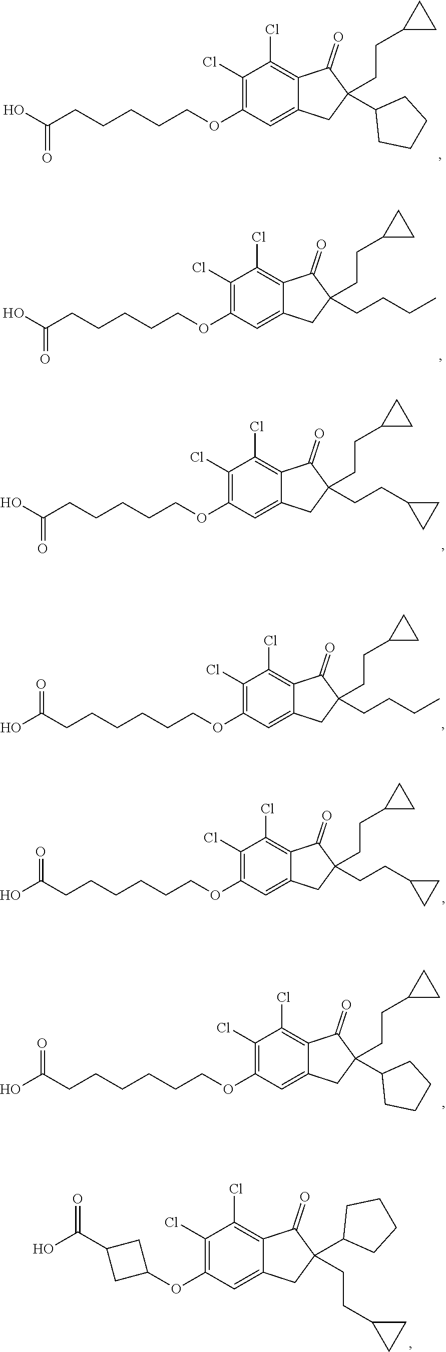

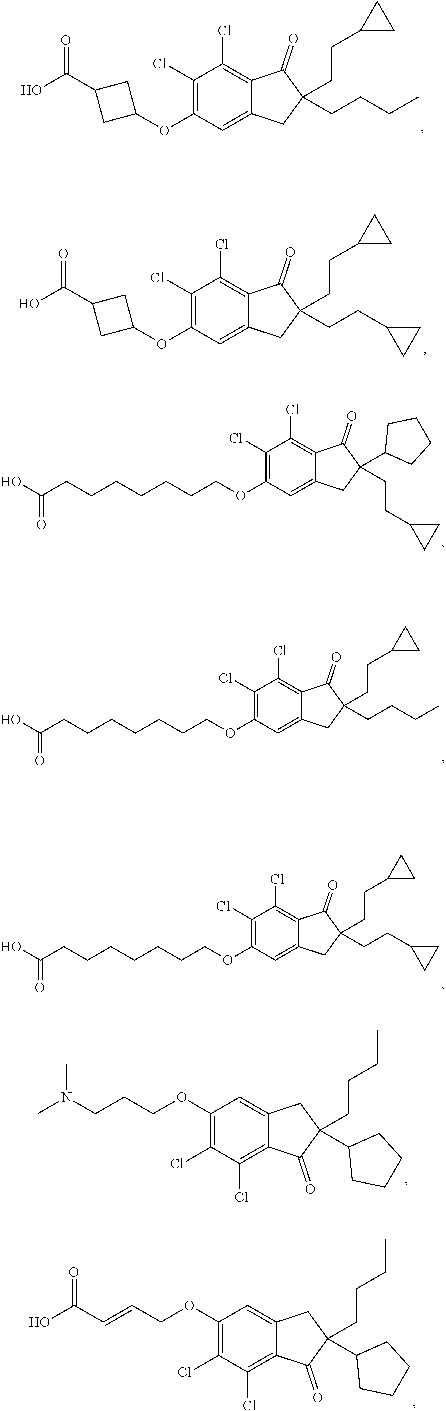

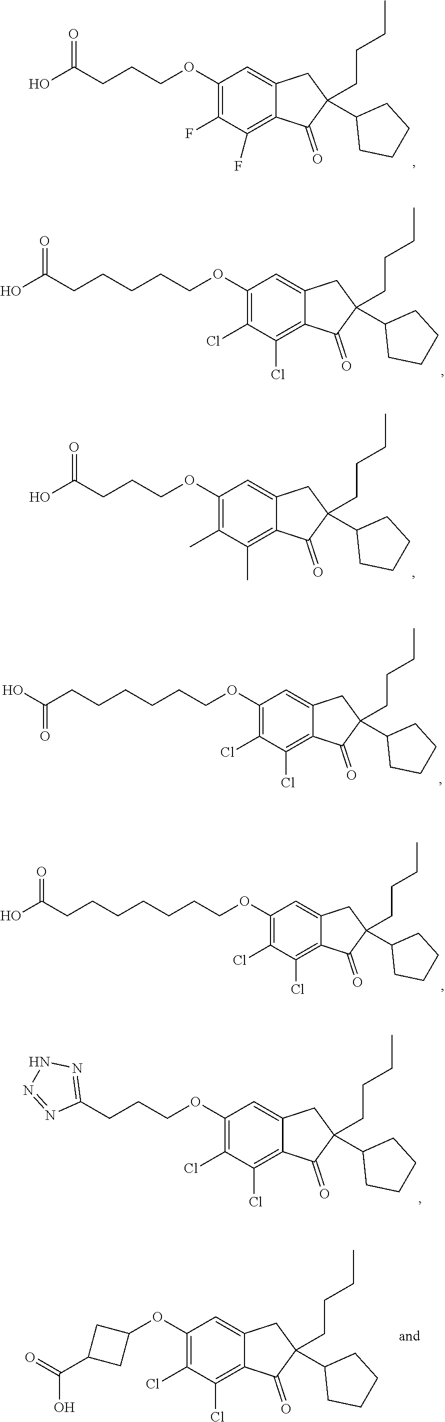

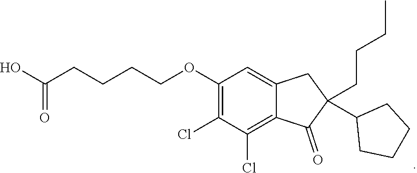 [SMILES: CCCCC1(C2CCCC2)Cc2cc(OCCCCC(=O)O)c(Cl)c(Cl)c2C1=O]

38. The method of any one of claims 10 to 37, wherein the compound modulates or inhibits an LRRC8 protein.

39. The method of claim 38, wherein the compound has a higher potency at modulating or inhibiting a SWELL1 channel than an equivalent amount of DCPIB (4-[2[butyl-6,7-dichloro-2-cyclopentyl-2,3-dihydro-1-oxo-1H-inden-5-yl)oxy]butanoic acid).

40. The method of any one of claims 1 to 39, wherein the administration of the compound is sufficient to upregulate the expression of LRRC8 or expression of an LRRC8-associated protein.

41. The method of any one of claims 1 to 40, wherein the administration of the compound is sufficient to stabilize LRRC8 protein complexes or an LRRC8-associated protein.

42. The method of any one of claims 1 to 41, wherein the administration of the compound is sufficient to promote membrane trafficking and activity of LRRC8 protein complexes or an LRRC8-associated protein.

43. The method of any one of claims 1 to 42, wherein the administration of the compound is sufficient to augment LRRC8-mediated signaling or trafficking to the membrane of intra-cellular organelles including but not limited to lysosomal-related organelles.
